(19) United States
(12) Patent Application Publication
(10) Pub. No.: US 2017/0165374 A1
(43) Pub. Date: Jun. 15, 2017

(54) COMPOSITIONS AND METHODS FOR TREATING BACTERIAL INFECTIONS

(71) Applicant: Insmed, Inc., Bridgewater, NJ (US)

(72) Inventors: Walter Perkins, Neshanic Station, NJ (US); Vladimir Malinin, Plainsboro, NJ (US); Franziska Leifer, Princeton, NJ (US); Carlos Figueroa, Maplewood, NJ (US); Keith DiPetrillo, Bridgewater, NJ (US)

(21) Appl. No.: 15/356,224

(22) Filed: Nov. 18, 2016

Related U.S. Application Data
(60) Provisional application No. 62/256,851, filed on Nov. 18, 2015.

(51) Int. Cl. A01N 25/00; A61K 31/7036; A61K 9/127

(52) U.S. Cl. CPC A61K 47/48807; A61K 47/48815; A61K 31/7036; A61K 9/127

(57) ABSTRACT

Provided herein are pharmaceutical compositions comprising an antibiotic encapsulated in liposomes. The lipid membrane component of the liposomes, or portion thereof comprises an unsaturated phospholipid. The antibiotic-to-lipid component weight ratio of the liposomes ranges from about 0.5-to-1 to about 3-to-1. The pharmaceutical compositions in some embodiments also include free antibiotic, in addition to encapsulated antibiotic. Methods for treating bacterial infections, e.g., pulmonary bacterial infections such as nontuberculous mycobacterial infections with the pharmaceutical compositions are also provided.

### CROSS REFERENCE TO RELATED APPLICATION

This application claims priority from U.S. Provisional Application Ser. No. 62/256,851, filed Nov. 18, 2015, the disclosure of which is incorporated herein for all purposes.

### BACKGROUND OF THE INVENTION

The incidences of infections caused by nontuberculous mycobacteria (NTM) have been reported to be growing. Faria et al. (2015). Journal of Pathogens, Article ID 809014. NTM infections can occur throughout the body, although skin and soft tissue, lymphadenitis and pulmonary infections are the most commonly described. Chan and Iseman (2013). Semin Respir Crit Care Med 34, pp. 110-23.

In addition to being able to form biofilms, which contributes to antibiotic resistance, NTM have been reported to reside and multiply in macrophages. In the case of pulmonary NTM, these bacteria can reside and multiply in macrophages in the airway submucosa as well as in alveolar macrophages. In instances where NTM reside and multiply in macrophages, in order for a treatment to be effective, pharmaceutical therapies should be designed to achieve bacteriostatic or bactericidal activity intracellularly (Rose et al. (2014). PLoS One 9(9), e108703. doi:10.1371/journal.pone.0108703).

Pulmonary NTM infection in the susceptible host can lead to potentially severe morbidity and even mortality among those affected. As infection rates are rising, pulmonary nontuberculous mycobacterial disease (PNTM) represents an emerging public health concern in the United States. The vast majority of pulmonary NTM infections in the United States are due to Mycobacterium avium complex (MAC), M. kansasii, M. abscessus, andM. fortuitum.

The prevalence of pulmonary NTM infections in the United States has more than doubled in the last 15 years. The ATS/IDSA PNTM reported 2-year period prevalence of pulmonary NTM infections is 8.6/100,000 persons. The prevalence of pulmonary NTM infections increases with age with 20.4/100,000 in those at least 50 years of age and is especially prevalent in females (median age: 66 years; female: 59%).

In the susceptible individual, pulmonary NTM infections can be serious or life threatening. Available therapies may be poorly tolerated, and may have significant adverse events.

The present invention addresses the need for effective treatments of bacterial infections, and specifically, effective treatments of pulmonary NTM infections, by providing novel antibiotic liposomal formulations and methods for using the same.

### SUMMARY OF THE INVENTION

Pathogens that are taken up intracellularly as well as those associated with biofilm formation such as certain species of Salmonella, Listeria and Mycobacterium represent a therapeutic challenge due to the requirement that antibiotics reach therapeutic levels at the intracellular site of infection and/or penetrate the biofilm. A consequence of this is that many antibiotics that are active in vitro are often inactive against the same bacterium in vivo. To account for this poor penetration, the present invention harnesses a liposomal delivery system designed to release its contents at the site of infection, e.g., by releasing liposomal contents upon lowering of pH or some other environmental factor. Alternatively or additionally, the liposomal membrane is designed to be dynamic so as to naturally release drug, e.g., by employing moderately stable liposomal bilayer.

The present invention is directed in one aspect, to a pharmaceutical composition comprising an antibiotic encapsulated in liposomes, e.g., an aminoglycoside or pharmaceutically acceptable salt thereof encapsulated in liposomes and methods for using the same. The lipid component of the liposomes comprises an unsaturated phospholipid and the antibiotic-to-lipid component weight ratio in the composition is from about 0.5-to-1 (antibiotic-to-lipid component) to about 3-to-1 (antibiotic-to-lipid component), e.g., from about 1-to-1 (antibiotic-to-lipid component) to about 3-to-1 (antibiotic-to-lipid component) or about from about 1.5-to-1 (antibiotic-to-lipid component) to about 3-to-1 (antibiotic-to-lipid component). Such compositions are administered to patients in need of the treatment of bacterial infections and specifically pulmonary bacterial infections. In some embodiments, the infections treatable with the pharmaceutical compositions described herein are pulmonary NTM infections, and the pharmaceutical compositions are delivered via inhalation to patients in need thereof. Further embodiments include the treatment of NTM abscessus pulmonary infections.

In one embodiment, the lipid component of the liposomes comprises an unsaturated phosphatidylethanolamine (PE), oleic acid, cholesteryl hemisuccinate (CHEMS), or a combination thereof. In one embodiment, the lipid component of the liposome comprises one of the lipid components provided in Table 2, Table 3 or Table 4.

For example, the lipid component of the liposomes in one embodiment, comprises an unsaturated phospholipid selected from 1,2-dioleoyl-sn-glycero-3-phosphocholine (DOPC), 1-palmitoyl-2-oleoyl-sn-glycero-3-phosphocholine (POPC), dioleoylphosphatidylethanolamine (DOPE), N-acyl phosphatidylethanolamine (NAPE) or a combination thereof. In a further embodiment, the lipid component of the liposomes comprises cholesterol, D-α-tocopherol-hemisuccinate (THS), cholesteryl hemisuccinate (CHEMS), Dipalmitoyl-sn-glycero-3-phosphoglycerol sodium (DPPG-Na) or a combination thereof. In one embodiment, the lipid component of the liposomes is one of the following: (i) DOPC; (ii) POPC; (iii) DOPC and DPPG-Na; (iv) POPE, cholesterol and DOPC; (v) POPE, cholesterol, THS and DOPC or (vi) POPE, CHEMS and DOPC.

In one embodiment, the encapsulated antibiotic in the pharmaceutical composition is an aminoglycoside or a pharmaceutically acceptable salt thereof. In a further embodiment, the encapsulated aminoglycoside is amikacin or a pharmaceutically acceptable salt thereof. In even a further embodiment, the pharmaceutically acceptable salt of amikacin is amikacin sulfate.

In one embodiment, the pharmaceutical composition provided herein includes a liposomally encapsulated aminoglycoside and the aminoglycoside is amikacin, streptomycin, or a pharmaceutically acceptable salt thereof. In even a further embodiment, the aminoglycoside is amikacin sulfate or streptomycin sulfate. In another embodiment, the composition comprises a liposomally encapsulated aminoglycoside, or a pharmaceutically acceptable salt thereof, and the aminoglycoside is apramycin, arbekacin, astromicin, capreomycin, dibekacin, framycetin, gentamicin, hygromycin B, isepamicin, kanamycin, neomycin, netilmicin, paromomycin, rhodestreptomycin, ribostamycin, sisomicin, spectinomycin, tobramycin, verdamicin, a pharmaceutically acceptable salt thereof, or a combination thereof. In another embodiment, the encapsulated aminoglycoside is AC4437, amikacin, apramycin, arbekacin, astromicin, bekanamycin, boholmycin, brulamycin, capreomycin, dibekacin, dactimicin, etimicin, framycetin, gentamicin, H107, hygromycin, hygromycin B, inosamycin, K-4619, isepamicin, KA-5685, kanamycin, neomycin, netilmicin, paromomycin, plazomicin, ribostamycin, sisomicin, rhodestreptomycin, sorbistin, spectinomycin, sporaricin, streptomycin, tobramycin, verdamicin, vertilmicin, or a pharmaceutically acceptable salt thereof.

In one embodiment, the antibiotic-to-lipid component weight ratio of the liposomal antibiotic is about 0.5-to-1 (antibiotic-to-lipid component) to about 3-to-1 (antibiotic-to-lipid component). For example, compositions of the invention include antibiotic and a lipid component at a weight ratio from about 1-to-1 (antibiotic-to-lipid component) to about 3-to-1 (antibiotic-to-lipid component); from about 1.25-to-1 (antibiotic-to-lipid component) to about 3-to-1 (antibiotic-to-lipid component); from about 1.5-to-1 (antibiotic-to-lipid component) to about 3-to-1 (antibiotic-to-lipid component); from about 1.75-to-1 (antibiotic-to-lipid component) to about 3-to-1 (antibiotic-to-lipid component); or from about 2-to-1 (antibiotic-to-lipid component) to about 3-to-1 (antibiotic-to-lipid component).

In one embodiment, the concentration of the antibiotic in the pharmaceutical composition is about 10 mg/mL or greater, e.g., from about 30 mg/mL to about 200 mg/mL. In a further embodiment, the concentration of the antibiotic in the pharmaceutical composition is about 20 mg/mL or greater. In a further embodiment, the concentration of the antibiotic in the pharmaceutical composition is about 30 mg/mL or greater, for example about 40 mg/mL to about 120 mg/mL or from about 40 mg/mL to about 80 mg/mL. In a further embodiment, the antibiotic is an aminoglycoside, e.g., an aminoglycoside selected from an aminoglycoside provided in Table 1, or a pharmaceutically acceptable salt thereof. In even a further embodiment, the aminoglycoside is amikacin, or a pharmaceutically acceptable salt thereof (e.g., amikacin sulfate).

In another embodiment of the pharmaceutical compositions described herein, a pharmaceutical composition is provided that includes both liposomally encapsulated antibiotic and free antibiotic. The encapsulated antibiotic and free antibiotic can be the same, or different. In one embodiment, both the liposomally encapsulated antibiotic and free antibiotic are aminoglycosides, or pharmaceutically acceptable salts thereof. In a further embodiment, the aminoglycosides, or pharmaceutically acceptable salts thereof, are each amikacin sulfate. The ratio by weight of free antibiotic to the antibiotic encapsulated liposomes, in one embodiment, is from about 1:100 to about 100:1, from about 1:50 to about 50:1, from about 1:10 to about 10:1, from about 1:5 to about 5:1, from about 1:4 to about 4:1, from about 1:3 to about 3:1, or from about 1:2 to about 2:1.

In another aspect of the invention, a method is provided for treating a bacterial infection, or a disease associated with a bacterial infection. The bacterial infection in one embodiment is a pulmonary bacterial infection. The bacterium in some embodiments is associated with a biofilm or resides intracellularly, or a combination thereof. The bacterial pulmonary infection in one embodiment is a mycobacterial infection. The mycobacterial infection in one embodiment is M. tuberculosis or M. leprae. The M. tuberculosis in one embodiment is multi-drug resistant. Multi-drug-resistant tuberculosis is also referred to as Vank's disease.

In one embodiment, the mycobacterial infection is an NTM infection. In a further embodiment, the pulmonary NTM infection is M. abscessus. The method for treating the bacterial infection comprises, in one embodiment, administering to a patient in need thereof, an effective amount of one of the pharmaceutical compositions described herein. In the case of a pulmonary bacterial infection, the administering comprises, in one embodiment, inhalation administration via an aerosol delivery device such as a nebulizer.

In one embodiment of the treatment methods provided herein, a method is provided for treating a bacterial pulmonary infection, or a disease associated with a pulmonary bacterial infection (pulmonary (respiratory) disease), e.g., bronchiectasis or chronic obstructive pulmonary disorder (COPD). In a further embodiment, the method comprises administering to the patient an effective amount of one of the liposomal aminoglycoside compositions described herein via inhalation delivery, for an administration period. In a further embodiment, the inhalation administration comprises inhalation administration via a nebulizer. In another embodiment, inhalation administration is via a dry powder inhaler (DPI).

In yet another embodiment, treatment methods provided herein comprises intravenous, subcutaneous, intranasal, intratracheal or intramuscular administration of an effective amount of one of the pharmaceutical compositions described herein to a patient in need thereof.

In embodiments where an effective amount of a composition described herein is administered via nebulization, the percent liposomal encapsulated antibiotic post-nebulization is from about 50% to about 95%, from about 50% to about 80%, from about 50% to about 75%, from about 50% to about 70%, from about 55% to about 75%, or from about 60% to about 70%. In a further embodiment, the aminoglycoside is an aminoglycoside provided in Table 1, or a pharmaceutically acceptable salt thereof. In a further embodiment, the aminoglycoside is amikacin, streptomycin or a pharmaceutically acceptable salt thereof. In even a further embodiment, the pharmaceutically acceptable salt of the aminoglycoside is amikacin sulfate.

In one embodiment, the treatment methods provided herein comprise aerosolizing the liposomal aminoglycoside composition and administering an effective amount of the aerosolized composition to a patient in need of treatment of a pulmonary NTM infection. The method in this embodiment therefore entails generation of an aerosolized liposomal aminoglycoside composition. Accordingly, in another aspect of the invention, an aerosolized liposomal aminoglycoside composition is provided. In one embodiment, the aerosolized composition comprises aerosol droplets having a size range of from about 1.0 μm to about 5.0 μm, from about 2.0 μm to 5.0 μm, from about 4.0 μm to about 5.0 μm, or from about 4.5 μm to about 5 μm. In a further embodiment, the aminoglycoside is an aminoglycoside set forth in Table 1, or a pharmaceutically acceptable salt thereof. In even a further embodiment, the aminoglycoside is amikacin, streptomycin or a pharmaceutically acceptable salt thereof (e.g., amikacin sulfate or streptomycin sulfate).

The pulmonary infection treatable by the compositions and methods provided herein in one embodiment is a pulmonary NTM infection. The pulmonary NTM infection in a further embodiment is M. abscessus. Treatment of such infections in one embodiment comprises administering to the patient in need thereof an effective amount of one of the liposomal aminoglycoside compositions provided herein via inhalation. In one embodiment, an aerosolized composition is generated and the aerosol is administered to the patient. The patient in need of treatment, in one embodiment, is a bronchiectasis patient, a cystic fibrosis patient, a patient that suffers from asthma or suffers from chronic obstructive pulmonary disorder (COPD).

In one embodiment, the NTM infection treatable by the methods and compositions provided herein is a pulmonary NTM infection selected from an M. avium, M. avium subsp. hominissuis (MAH), M. abscessus, M. chelonae, M. bolletii, M. kansasii, M. ulcerans, M. avium, M. avium complex (MAC) (M. avium and M. intracellulare), M. conspicuum, M. peregrinum, M. immunogenum, M. xenopi, M. massiliense, M. marinum, M. malmoense, M. mucogenicum, M. nonchromogenicum, M. scrofulaceum, M. simiae, M. smegmatis, M. szulgai, M. terrae, M. terrae complex, M. haemophilum, M. genavense, M. gordonae, M. ulcerans, M. fortuitum, M. fortuitum complex (M. fortuitum and M. chelonae) infection or a combination thereof. In a further embodiment, the pulmonary NTM infection is a M. abscessus infection. Other embodiments include the treatment of intracellular pulmonary NTM infections M. massiliense, M. kansasii, M. fortuitum, M. chelonae, M. xenopi or M. simiae, or a combination thereof. In one embodiment, the NTM infection is a pulmonary recalcitrant NTM infection. The NTM treatment methods provided herein in one embodiment result in a change from baseline on the semi-quantitative scale for mycobacterial culture for a treated patient, and/or NTM culture conversion to negative after the administration period. Culture conversion is defined as at least three consecutive monthly sputum samples that test negative for NTM bacteria. Testing for culture conversion can begin during the administration period. Methods provided herein can also be used to treat a disease associated with a pulmonary NTM infection, e.g., bronchiectasis.

Other bacterial infections amenable for treatment with the compositions and methods provided herein include Salmonella (e.g., Salmonella typhimurium, Salmonella typhi), Listeria (e.g., Listeria monocytogens, e.g., Listeria associated with meningitis and spesis) or Francisella, Streptobacillus (e.g., Streptobacillus moniliformis), Trypanosoma (e.g., Trypanosoma brucei, associated with sleeping sickness and nagana), Entamoeba (e.g., Entamoeba histolytica, Entamoeba dispar), Cryptosporidium (e.g., Cryptosporidium parvum) Coxiella burnetii (causative agent of Q fever), Streptococcus (e.g., Streptococcal L-forms; S. mutans, S. pyogenes; S. agalactiae), Porphyromonas (e.g., P. gingivalis), Eikenella corrodens, Prevotella (e.g., Prevotella melaninogenica, Prevotella intermedia), Chlamydia (e.g., Chlamydia trachomatis), Tannerella forsythia, Treponema (e.g., Treponema denticola, Treponema palladium, Treponema carateum), Mycoplasma (M. genitalium, M. pneumoniae), Yersina (Y. pestis, Y. aldovae, Y. aleksiciae, Y. bercovien, Y. enterocolitica, Y. entomophaga, Y. frederiksenii, Y. intermdia, Y. kristensenii, Y. massiliensis, Y. mollaretii, Y. nurmii, Y. pekkanenii, Y. philomiragia, Y. pseudotuberculosis, Y. rohdei, Y. ruckeri, Y. similis), Corynebacterium (e.g., C. diphtheria), or a Borrelia infection. Other embodiments include treatment of Rhodococcus (e.g., R. equi and/or R. fascians) infections. In addition, diseases associated with the aforementioned bacterial infections are amenable for treatment with the methods provided herein.

In another embodiment, the bacterial infection is a malaria parasite infection, Shigellae (e.g., S. boydii, S. dysenteriae, S. flexneri, S. sonnei), L. pneumophila, Rickettsia, a Legionella bacteria such as L. pneumophila, L. longbeachae, L. feeleii, L. micdadei, L. anisa. Diseases associated with the aforementioned pathogens are also treatable with the methods described herein, e.g., Legionnaire's disease and Pontiac fever in the case of a Legionella infection; dysentery in the case of Shigella infection; typhus and other arthropod-borne diseases in the case of Rickettsia.

### BRIEF DESCRIPTION OF THE FIGURES

FIG. 1 is a bar graph showing percentage of liposome leakage for various compositions after incubation in media or cell culture supernatant. Concentrations refer to lipid concentrations.

FIG. 2 is a bar graph showing percentage of calcein release inside cells for various incubation times. Concentrations refer to lipid concentrations.

FIG. 3 is a bar graph showing the uptake of fluorescently labeled liposome formulations by differentiated THP-1 cells after 4 hour incubation (MFI).

FIG. 4 is a graph of intracellular CFU as a function of amikacin concentration after 4 day treatment with various liposomal amikacin formulations.

FIG. 5 is a graph of intracellular CFU as a function of amikacin concentration after 4 day treatment with POPE-based amikacin liposomal formulations.

FIG. 6 is a bar graph showing the percent reduction of M. ab. NIH26 CFU after 4 day treatment with amikacin-loaded liposomal formulations. Percent reduction is calculated from a baseline of free amikacin treatment at 128 μg/mL, n=3.

FIG. 7 are graphs showing the percent reduction of M. ab. NIH26 CFU after 4 day treatment with amikacin-loaded liposomal formulations. Percent reduction is calculated from a baseline of free amikacin treatment at 32, 64, and 128 μg/mL, n=3.

FIG. 8 is a graph showing macrophage cell death (as a percent of healthy control) after four day treatment with various liposomal amikacin formulations.

FIG. 9 is a graph showing macrophage cell death (as a percent of healthy control) after four day treatment with POPE based liposomal amikacin formulations.

### DETAILED DESCRIPTION OF THE INVENTION

Fast-growing NTM subspecies give rise to chronic debilitating lung infections that are difficult to eradicate with existing antibiotic treatments. The present invention addresses this need by providing novel pharmaceutical compositions comprising a liposomally encapsulated antibiotic, designed to efficiently deliver an antibiotic payload to NTM biofilms and to infected phagocytic cells for subsequent intracellular release of the antibiotic, e.g., aminoglycoside. Families of liposomal antibiotic compositions Lipids used to form the liposomes described herein include unsaturated phospholipids such as 1-palmitoyl-2-oleoyl-sn-glycero-3-phosphoethanolamine (POPE) and unsaturated PC lipids, e.g., to harness moderately stable bilayer formation.

The present invention harnesses pharmaceutical compositions comprising liposomes made up of unsaturated phospholipids to allow for efficient delivery of the liposomal contents—e.g., intracellular release of one or more antibiotics such as one or more aminoglycosides or pharmaceutically acceptable salts thereof or release of antibiotic at a biofilm. Without wishing to be bound by theory, release of antibiotic is accomplished via harnessing a liposomal lipid membrane that is either dynamic, that exhibits a physical or electrical change upon a reduction in environmental pH (e.g., at acidic pH from about neutral pH), in response to a secondary factor, and/or a membrane that is fusogenic. In one embodiment, the antibiotic (e.g., aminoglycoside or pharmaceutically acceptable salt thereof) is released from inside the liposome during or after the physical or electrical change into the intracellular space of a cell, or at a biofilm surface, or released after penetrating a biofilm. The intracellular space includes in one embodiment, an endosome, phagosome or a phagolysosome or the cell cytosol. In one embodiment, the physical or electrical change induces the lipid to fuse with a target membrane, e.g., a cell membrane, endosome membrane, phagosome membrane, phagolysosome membrane, etc. Release of the antibiotic inside the cell's cytosol can follow fusion of the liposome membrane with an endosomal membrane, phagosomal membrane, phagolysosomal membrane or a lysosomal membrane. In one embodiment, where an environmental factor induces a physical or electrical change in the liposome structure, the environmental factor is calcium or an enzyme. In some embodiments, the secondary (environmental) factor is present inside a phagosome, phagolysosome or endosome or in a biofilm (or at the surface of a biofilm).

The interactions between the liposomal vesicles and cells can occur via one or more processes, such as stable physical adsorption, endocytosis, lipid exchange, and fusion. The use of one of the above mechanisms does not exclude the use of others, or a combination of mechanisms. Moreover, intracellular release of antibiotic does not exclude antibiotic release at or near a bacterial (e.g., NTM) biofilm.

In one aspect, the present invention is directed to a pharmaceutical composition comprising an antibiotic (e.g., aminoglycoside) or a pharmaceutically acceptable salt thereof encapsulated in liposomes. The liposomal lipid membrane component comprises an unsaturated phospholipid, and in various embodiments, is dynamic, pH sensitive and/or fusogenic. The pharmaceutical composition comprises a liposomal dispersion comprising liposomes comprising encapsulated antibiotic. Liposomal lipid components of the liposomes are discussed below. A “liposomal dispersion” refers to a solution or suspension comprising a plurality of liposomes. Also discussed below are embodiments that include both free antibiotic and liposomally encapsulated antibiotic in a pharmaceutical composition.

Liposomes are completely closed lipid bilayer membranes containing an entrapped aqueous volume. Liposomes in the composition may be unilamellar vesicles (possessing a single membrane bilayer), plurilamellar vesicles (liposomes within liposomes with irregular spacing between membranes), or multilamellar vesicles (onion-like structures characterized by multiple membrane bilayers, each separated from the next by an aqueous layer that tend to be regularly spaced) or a combination thereof. The liposomal bilayer is composed of two lipid monolayers having a hydrophobic “tail” region and a hydrophilic “head” region. The structure of the membrane bilayer is such that the hydrophobic (nonpolar) “tails” of the lipid monolayers orient toward the center of the bilayer while the hydrophilic “heads” orient towards the aqueous phase.

In one embodiment, the encapsulated antibiotic in the pharmaceutical composition is cationic. For example, the antibiotic in one embodiment is an aminoglycoside, a polymyxin (e.g., polysporin, neosporin, polymyxin B, colistin), a cationic steroid antibiotic (e.g., a ceragenin) or a cationic peptide antibiotic (e.g., an antibiotic described by Savage et al. (2002). FEMS Microbiology Letters 217, pp. 1-7; Bahar and Ren (2013). Pharmaceuticals (Basel) 6, pp. 1543-1575; each of which is incorporated by reference herein in its entirety for all purposes). The antibiotic in one embodiment, is an aminoglycoside or pharmaceutically acceptable salt thereof. For example, streptomycin, amikacin or a pharmaceutically acceptable salt thereof can be provided in one of the compositions provided herein.

In one embodiment, the liposomally encapsulated antibiotic is an aminoglycoside or pharmaceutically acceptable salt thereof. In a further embodiment, the aminoglycoside is amikacin, apramycin, arbekacin, astromicin, capreomycin, dibekacin, framycetin, gentamicin, hygromycin B, isepamicin, kanamycin, neomycin, netilmicin, paromomycin, rhodestreptomycin, ribostamycin, sisomicin, spectinomycin, streptomycin, tobramycin, verdamicin, or a pharmaceutically acceptable salt thereof. In a further embodiment, the aminoglycoside is amikacin. In even a further embodiment, the amikacin is amikacin sulfate. In another embodiment, the aminoglycoside is selected from an aminoglycoside set forth in Table 1, a pharmaceutically acceptable salt thereof, or a combination thereof. For example, a pharmaceutically acceptable salt such as a sulfate salt of one or more of the aminoglycosides set forth in Table 1 (or pharmaceutically acceptable salts of the same) can be formulated in liposomes and in a pharmaceutical composition, and administered to a patient in need of treatment of an intracellular bacterial infection, e.g., a pulmonary NTM infection, e.g., via pulmonary delivery by a nebulizer or a dry powder inhaler (DPI).

TABLE 1
Aminoglycosides for use with the present invention

| | | | |
|---|---|---|---|
| AC4437 | dibekacin | K-4619 | sisomicin |
| amikacin | dactimicin | isepamicin | rhodestreptomycin |
| arbekacin | etimicin | KA-5685 | sorbistin |
| apramycin | framycetin | kanamycin | spectinomycin |
| astromicin | gentamicin | neomycin | sporaricin |
| bekanamycin | H107 | netilmicin | streptomycin |
| boholmycin | hygromycin | paromomycin | tobramycin |
| brulamycin | hygromycin B | plazomicin | verdamicin |
| capreomycin | inosamycin | ribostamycin | vertilmicin |

In one embodiment, a pharmaceutical composition of the invention comprises a combination of aminoglycosides, or pharmaceutically acceptable salts thereof, e.g., a combination of two or more aminoglycosides, or pharmaceutically acceptable salts thereof, as set forth in Table 1 which are encapsulated in liposomes. In one embodiment, the composition comprising the liposomal encapsulated aminoglycoside comprises from 1 to about 6 aminoglycosides, or pharmaceutically acceptable salts thereof. In another embodiment, the composition comprising the liposomal encapsulated aminoglycoside comprises at least 1, at least 2, at least 3, at least 4, at least 5, or at least 6, of the aminoglycosides set forth in Table 1 (or pharmaceutically acceptable salts of the aminoglycosides). In another embodiment, a pharmaceutical composition provided herein comprises between 1 and 4 aminoglycosides, or pharmaceutically acceptable salts thereof. In a further embodiment, the combination comprises amikacin or streptomycin, e.g., as amikacin sulfate or streptomycin sulfate.

In one embodiment, the aminoglycoside is an aminoglycoside free base, or its salt, solvate, or other non-covalent derivative. In a further embodiment, the aminoglycoside is amikacin. Included as suitable aminoglycosides used in the drug compositions of the present invention are pharmaceutically acceptable addition salts and complexes of drugs. In cases where the compounds may have one or more chiral centers, unless specified, the present invention comprises each unique racemic compound, as well as each unique nonracemic compound. In cases in which the active agents have unsaturated carbon-carbon double bonds, both the cis (Z) and trans (E) isomers are within the scope of this invention. In cases where the antibiotic exists in tautomeric forms, such as keto-enol tautomers, each tautomeric form is contemplated as being included within the invention. For example, amikacin, in one embodiment, is present in the pharmaceutical composition as amikacin base, or amikacin salt, for example, amikacin sulfate or amikacin disulfate. In another embodiment, streptomycin base or a pharmaceutically acceptable streptomycin salt is present in the pharmaceutical composition.

Antibiotics used in the pharmaceutical compositions provided herein can also be deuterated at one or more hydrogens.

In a further embodiment, the liposomally encapsulated aminoglycoside is present in the pharmaceutical composition at a concentration of about 10 mg/mL aminoglycoside or greater, about 20 mg/mL aminoglycoside or greater, about 30 mg/mL aminoglycoside or greater, about 40 mg/mL aminoglycoside or greater, about 50 mg/mL aminoglycoside or greater, about 60 mg/mL aminoglycoside or greater, or about 70 mg/mL aminoglycoside or greater, or about 80 mg/mL aminoglycoside or greater. In a further embodiment, the liposomally encapsulated aminoglycoside is amikacin or streptomycin, e.g., amikacin sulfate or streptomycin sulfate. In another embodiment, the liposomally encapsulated aminoglycoside is present in the pharmaceutical composition at a concentration of from about 10 mg/mL to about 150 mg/mL aminoglycoside, from about 20 mg/mL to about 100 mg/mL aminoglycoside, from about 30 mg/mL to about 90 mg/mL aminoglycoside or from about 40 mg/mL to about 90 mg/mL aminoglycoside.

The present invention is based in part on the use of a pharmaceutical composition comprising liposomally encapsulated antibiotic that allows for the delivery of the liposomal contents intracellularly or to a bacterial biofilm (e.g., either penetration of the biofilm or delivery at the surface of the biofilm). Various lipid membrane components are useful to achieve this goal. Without wishing to be bound to theory, it is thought that the compositions of the present invention achieve liposomal release of antibiotic by one or more mechanisms. In one embodiment, the liposomal lipid membrane undergoes a structural or electrical change. The structural or electrical change can be due to a property of the liposome's environment. For example, in one embodiment, the lipid membrane component of the liposome, or a portion thereof, undergoes a physical (e.g., structural) or electrical charge change under acidic conditions (e.g., due to a pH drop in the liposome's environment), or in the presence of a secondary factor such as calcium or an enzyme. The secondary factor, in one embodiment, is present in an endosome, lysosome, phagosome or a phagolysosome. Alternatively or additionally, the structural change can be attributed to the use of one or more moderately stable liposomal lipid membrane components, e.g., to provide a dynamic membrane that allows for release of liposome contents.

The structural and/or electrical change results in a leakage of liposome contents and/or the promotion of a fusion event of the liposome with a target membrane, for example, a eukaryotic plasma membrane, or an endosome, phagosome, phagolysosome, or lysosome membrane. Liposomes that are able to fuse with a target membrane are referred to herein as “fusogenic”. Liposomes that are able to release contents in response to a secondary factor such as pH or an enzyme are referred to herein as “triggerable” liposomes. Both fusogenic and triggerable liposomes are within the scope of the present invention.

In one embodiment, release of the antibiotic occurs inside the cell's cytosol following fusion of the liposome membrane with an endosomal membrane, phagosomal membrane, phagolysosomal membrane or a lysosomal membrane.

In one embodiment, the liposome is a “triggerable” liposome and the liposome releases its contents inside an endosome, a lysosome, a phagosome, a phagolysosome with subsequent membrane fusion and cytosolic release, or at a bacterial biofilm.

pH sensitive liposomes are stable at physiological pH but undergo destabilization under acidic conditions (pH drop), for example, conditions present in the endosome, phagosome, or phagolysosome lumen or the acidic environment of inflamed, infected and/or tumorigenic tissue. A result of the lipid component of the liposome being able to undergo a transition under acidic conditions (or some other environmental condition), the internal contents of the liposome is released into the environment. In the case of a fusion event with a cell, release of liposome contents results in intracellular delivery of the liposome payload. Various fusogenic and/or triggerable liposome lipid components and strategies for producing the same are provided herein.

In another embodiment, the liposome harnesses a “dynamic” lipid membrane (e.g., without cholesterol or saturated-chain lipids whose main phase transition temperature is above 37 C), to allow for release of contents. An example of this embodiment includes a lipid membrane component comprising an unsaturated phospholipid. The phospholipid, in one embodiment is an unsaturated phosphatidylcholine. In a further embodiment, the phosphatidylcholine is 1,2-dioleoyl-sn-glycero-3-phosphocholine (DOPC).

The compositions described herein find utility in treating a number of intracellular infections, including pulmonary NTM infections, as described further below.

Each liposome in the pharmaceutical composition provided herein has a liposomal membrane comprising one or more lipids (referred to herein as a “lipid component” of a liposome). The lipid component in one embodiment, comprises a synthetic lipid, semi-synthetic lipid, a naturally-occurring lipid, or a combination thereof. In addition, net neutral, cationic and/or anionic lipids can be used as a lipid in a lipid component.

The lipid component of the liposomes in one embodiment comprises 1,2-dioleoyl-sn-glycero-3-phosphocholine (DOPC), 1-palmitoyl-2-oleoyl-sn-glycero-3-phosphocholine (POPC), dioleoylphosphatidylethanolamine (DOPE), N-acyl phosphatidylethanolamine (NAPE) or a combination thereof. In a further embodiment, the lipid component of the liposomes comprises cholesterol, D-α-tocopherol-hemisuccinate (THS), cholesteryl hemi succinate (CHEMS), Dipalmitoyl-sn-glycero-3-phosphoglycerol sodium (DPPG-Na) or a combination thereof.

In one embodiment, the lipid component of the liposomes consists of one of the following: (i) DOPC; (ii) POPC; (iii) DOPC and DPPG-Na; (iv) POPE, cholesterol and DOPC; (v) POPE, cholesterol, THS and DOPC or (vi) POPE, CHEMS and DOPC.

In one embodiment, the lipid component comprises an unsaturated phosphatidylethanolamine (PE). For example, the unsaturated PE in one embodiment is dioleoylphosphatidylethanolamine (DOPE), N-acyl phosphatidylethanolamine (NAPE) or 1-palmitoyl-2-oleoyl-sn-glycero-3-phosphoethanolamine (POPE). In a further embodiment, the unsaturated PE is palmitoyl-2-oleoyl-sn-glycero-3-phosphoethanolamine (POPE). In even a further embodiment, the lipid component of the liposomes comprise POPE and either cholesterol or CHEMS.

The unsaturated PEs do not form bilayers on their own, i.e., as single lipid components of a liposome. Instead, the unsaturated PE can be stabilized into liposomes through the addition of lipids that favor a bilayer structure. In one liposome embodiment, the lipid component comprises an unsaturated PE and 1,2-dioleoyl-sn-glycero-3-phosphocholine (DOPC) or oleic acid (OA). Liposomal lipid components comprising an unsaturated PE and oleic acid in another embodiment comprise cholesterol. For example, the unsaturated PE and oleic acid can be present in a 2:1 molar ratio. In another embodiment, an unsaturated PE, oleic acid and cholesterol are present in a molar ratio of 2:1:2 ratio. A liposomal lipid component comprising an unsaturated PE in another embodiment includes cholesterol and didodecyldimethlyammonium (DODAC) and optionally further includes phosphatidylethanolamine (PE)-PEG. Various liposomal lipid components comprising unsaturated PEs and other components are provided in Table 2, Table 3 and Table 4, and are amenable for use with the compositions and methods described herein.

Other combinations of lipids for use herein include but are not limited to DOPE/OA, DOPE/palmitoylhomocysteine (PHC), DOPE/dipalmitoylsuccinylglycerol (DSPG) and DOPE/cholesteryl hemisuccinate (CHEMS).

In another liposome embodiment, the lipid component comprises DOPE and dioleoylphosphatidylserine (DOPS). In yet another embodiment, the lipid component of the liposome comprises DOPE and N-succinyl-DOPE or N-glutaryl-DOPE. Without wishing to be bound by theory, it is thought that the use of a lipid in the liposome having a negatively charged head group, acidification results in neutralizing the lipid charge which reduces liposome bilayer stability. This decrease in stability of the liposomal bilayer can lead to fusion of the liposome to a target membrane and/or antibiotic release from the liposome.

In one embodiment, one of the lipid combinations described by Lutwyche is amenable for use as liposomal lipid components in the pharmaceutical compositions and methods described herein (Lutwyche et al. (1998). Antimicrobial Agents and Chemotherapy 42, pp. 2511-2520, incorporated by reference herein in its entirety for all purposes). In one embodiment, the lipid component is selected from one of the lipid components in Tables 2-4 (and combinations thereof).

In one embodiment the lipid component comprises DOPE, N-succinyl-DOPE and PEG-ceramide. In a further embodiment, the molar ratio of the DOPE/N-succinyl-DOPE/PEG-ceramide lipid component is 69.5:30:0.5 or 65:30:5. In another embodiment the lipid component comprises DOPE, N-succinyl-DOPE, cholesterol and PEG-ceramide. In a further embodiment, the molar ratio of the DOPE/N-succinyl-DOPE/cholesterol/PEG-ceramide lipid component is 49.5:30:20:0.5 or 45:30:20:5.

A lipid component of the liposome in one embodiment is one of the components described by Simöes (Simöes et al. (2004). Advanced Drug Delivery Reviews 56, pp. 947-965, incorporated by reference herein in its entirety).

The lipid component of the liposome in one embodiment comprises cholesteryl hemisuccinate (CHEMS). In a further embodiment, the lipid component comprises DOPE. In another embodiment, the lipid component of the liposome comprises N-acyl phosphatidylethanolamine (NAPE), 1,2-Dioleoyloxy-3-dimethylaminopropane (DODAP) and 1,2-Dioleoyl-sn-glycero-3-phosphocholine (DOPC). In yet another embodiment, the lipid component of the liposome comprises 1-palmitoyl-2-oleoyl-sn-glycero-3-phosphoethanolamine (POPE), cholesterol and D-α-tocopherol-hemisuccinate (THS).

The lipid membrane component of the liposome, in one embodiment, include oleyl alcohol (OAlc) in combination with a phosphatidylcholine (PC). Without wishing to be bound by theory, it is thought that OAlc is capable of forming a hydrogen bond through its hydroxyl to an oxygen atom on the phosphate group on the PC, resulting in a complex with geometry similar to that of DOPE. Potentially, this results in a lowering of the energy barrier for the lipid transition from a lamellar phase to a hexagonal II phase, which is implicated in membrane destabilization (see, e.g., Sudimack et al. (2002). Biochimica et Biophysica Acta 1564 pp. 31-37, incorporated by reference herein in its entirety for all purposes).

TABLE 2
Liposomal Lipid Component Embodiments

| | Optional Molar |
|---|---|
| Lipid Component | Ratios |
| PE/CHEMS | |
| PE/PC/CHEMS | 4:2:4 to 1:4:4 |
| PE/Chol | |
| PE/THS | |
| PE/CHEMS/Chol | 7:4:2 |
| DOPE/OA/Chol | 1:1.3:0.4 |
| DOPE/CHEMS | 6:4; 2:1 |
| NAPE/CHEMS | 6:4; 2:1 |
| DOPE/Chol | |
| POPE/Chol | |
| NAPE/Chol | |
| DOPE/Chol/THS | 4:4:1 |
| POPE/Chol/THS | 4:4:1 |
| NAPE/Chol/THS | 4:4:1 |
| POPE/CHEMS | 3:2; 2:1 |
| DOPE/CHEMS | 3:2 |
| DOPE/CHEMS/Chol | |
| DOPE/DODAP/DOPC | 2:2:1 |
| NAPE/DODAP/DOPC | 2:2:1 |
| POPE/DODAP/DOPC | 2:2:1 |
| DOPE/DOPS/PEG-ceramide | |
| NAPE/DOPS/PEG-ceramide | |
| POPE/DOPS/PEG-ceramide | |
| DOPE/N-succinyl-DOPE | 7:3 |
| DOPE/N-succinyl-DOPE/PEG-ceramide | 69.5:30:0.5 |
| | 65:30:5 |
| DOPE/N-succinyl-DOPE/Chol/PEG-ceramide | 7:6:6:1 |
| | 39.5:30:30:0.5 |
| | 49.5:30:20:0.5 |
| | 45:30:20:5 |
| DOPE/N-glutaryl-DOPE | 7:3 |
| NAPE/N-glutaryl-DOPE | 7:3 |
| POPE/N-glutaryl-DOPE | 7:3 |
| DOPE/N-glutaryl-DOPE/PEG-ceramide | 69.5:30:0.5 |
| | 65:30:5 |
| DOPE/N-glutaryl-DOPE/Chol/PEG-ceramide | 7:6:6:1 |
| | 39.5:30:30:0.5 |
| | 49.5:30:20/0.5 |
| | 45:30:20:5 |
| DOPE/DSPG/DSPE-PEG | 7:3:5 |
| DOPE/DOSG | 1:1 |
| NAPE/DOSG | 1:1 |
| POPE/DOSG | 1:1 |
| DOPE/HSPC/CHEMS/Chol | 1:1:1:1 |
| DOPE/HSPC/CHEMS/Chol | 2:1:1:1 |
| EPC/DDAB/CHEMS/Tween-80 | 25:25:49:1 |
| PC/DDAB/CHEMS/Tween-80 | 25:25:49:1 |
| PC/CHEMS/Tween-80/OAlc | 10:10:1:16 |
| | 25:25:1:40 |
| PC/CHEMS/Tween-80/OAlc | 25:25:1:40 |
| DOPE/N-citraconyl-DOPE/Chol | 45.8:10:40 |
| NAPE/N-citraconyl-DOPE/Chol | 45.8:10:40 |
| POPE/N-citraconyl-DOPE/Chol | 45.8:10:40 |
| DDAB/CHEMS | 7:3; 3:7 |
| POPE/Chol/MPL | |
| YSK05/POPE/Cholesterol/DMG-PEG | 50:25:25:3 |
| Diolein/CHEMS | 3:2 |
| EPC/CHEMS/T-80/OAlc | 10:10:1:16 |
| EPC/CHEMS/DDAB/T-80 | 25:49:25:1 |

CHEMS: cholesteryl hemisuccinate;
Chol: cholesterol;
DDAB: dimethyldioctadecylammonium bromide;
DMG: dimyristoylglycerol;
DODAP: 1,2-Dioleoyloxy-3-dimethylaminopropane;
DOPC: 1,2-Dioleoyl-sn-glycero-3-phosphocholine;
DOPE: dioleoylphosphatidylethanolamine;
DOSG: dioleylsuccinylglycerol;
DPPC: dipalmitoylphosphatidylcholine;
DSPG: dipalmitoylsuccinylglycerol;
EPC: egg yolk phosphatidylcholine;
MPL: monophosphoryl lipid A;
NAPE: N-acyl phosphatidylethanolamine;
OA: oelic acid;
OAlc: oleyl alcohol;
PE: phosphatidylethanolamine;
PEG: polyethylene glycol;
POPE: palmitoyloleoylglycero phosphoethanolamine;
THS: D-α-tocopherol-hemisuccinate;
YSK05 (synthetic pH sensitive lipid) https://www.ncbi.nlm.nih.gov/pubmed/24727060

TABLE 3
Liposomal Lipid Component Embodiments

| # | Lipid 1 | Lipid 2 | Lipid 3 | Lipid 4 |
|---|---|---|---|---|
| 1 | POPE | CHEMS | Chol | — |
| 2 | DOPE | CHEMS | Chol | — |
| 3 | DOPE | CHEMS | | — |
| 4 | POPE | Chol | THS | — |
| 5 | NAPE | DODAP | DOPC | — |
| 6 | POPC | CHEMS | OAlc | — |
| 7 | diolein | CHEMS | — | — |
| 8 | DOPC | — | — | — |
| 9 | DOPE | Oleic Acid | — | — |
| 10 | DOPE | DOPC | — | — |
| 11 | Chol | DOPC | DPPS | — |
| 12 | DOPE | CHEMS | DOPC | — |
| 13 | DOPE | CHEMS | DOPC | — |
| 14 | POPE | Chol | THS | DOPC |
| 15 | POPE | Chol | THS | DOPC |
| 16 | POPE | Chol | THS | POPC |
| 17 | POPE | Chol | THS | — |
| 18 | DOPE | CHEMS | POPC | — |
| 19 | DPPC | DPPG | | — |
| 20 | DOPE | CHEMS | DOPC | — |
| 21 | POPE | Chol | THS | DOPC |
| 22 | DOPC | CHEMS | — | — |
| 23 | DOPC | THS | — | — |
| 24 | DOPC | DPPG-Na | — | — |
| 25 | POPE | CHEMS | DOPC | — |
| 26 | POPE | Chol | DOPC | — |
| 27 | POPE | THS | DOPC | — |
| 28 | POPC | — | — | — |
| 29 | POPE | — | — | — |
| 30 | DOPE | — | — | — |
| 31 | DLinPC | — | — | — |
| 32 | POPE | CHEMS | DOPC | — |
| 33 | POPE | THS | DOPC | — |
| 34 | DOPC | THS | — | — |

POPE: 1-palmitoyl-2-oleoyl-sn-glycero-3-phosphoethanolamine;
Chol: cholesterol;
DOPC: 1,2-dioleoyl-sn-glycero-3-phosphocholine;
CHEMS: cholesterol hemi-succinate;
THS: tocopherol hemisuccinate;
DPPG-Na: 1,2-Dipalmitoyl-sn-glycero-3-phosphoglycerol, sodium salt;
POPC: 1-palmitoyl-2-oleoyl-sn-glycero-3-phosphocholine;
DPPC: 1,2-dipalmitoyl-sn-glycero-3-phosphocholine;
DlinPC: 1,2-dilinoleoyl-sn-glycero-3-phosphocholine;
DOPE: 1,2-dioleoyl-sn-glycero-3-phosphoethanolamine;
NAPE: N-acetyrphosphatidylethanolamine;
DODAP: 1,2-dioleoyl-3-dimethylammonium-propane

TABLE 4
Liposomal Lipid Component Embodiments

| | Lipid | | | | Molar Ratio | | | |
|---|---|---|---|---|---|---|---|---|
| | Lipid | Lipid | Lipid | Lipid | Lipid | Lipid | Lipid | Lipid |
| # | 1 | 2 | 3 | 4 | 1 | 2 | 3 | 4 |
| 3 | DOPE | CHEMS | — | — | 3 | 2 | — | — |
| 4 | POPE | Chol | THS | — | 4 | 4 | 1 | — |
| 5 | NAPE | DODAP | DOPC | — | 2 | 2 | 1 | — |
| 6 | POPC | CHEMS | OAlc | — | 5 | 5 | 8 | — |
| 7 | diolein | CHEMS | — | — | 3 | 2 | — | — |
| 8 | DOPC | — | — | — | 1 | — | — | — |
| 9 | DOPE | Oleic | — | — | 7 | 3 | — | — |
| | | Acid | | | | | | |
| 10 | DOPE | DOPC | — | — | 3 | 2 | — | — |
| 11 | Chol | DOPC | DPPS | — | 5 | 4 | 1 | — |
| 12 | DOPE | CHEMS | DOPC | — | 12 | 8 | 5 | — |
| 13 | DOPE | CHEMS | DOPC | — | 9 | 6 | 10 | — |
| 14 | POPE | Chol | THS | DOPC | 8 | 8 | 2 | 5 |
| 15 | POPE | Chol | THS | DOPC | 12 | 12 | 3 | 20 |
| 16 | POPE | Chol | THS | POPC | 8 | 8 | 2 | 5 |
| 18 | DOPE | CHEMS | POPC | — | 12 | 8 | 5 | — |
| 19 | DOPE | CHEMS | POPC | — | 9 | 6 | 10 | — |
| 20 | DPPC | DPPG | | — | 19 | 1 | | — |
| 21 | DOPE | CHEMS | DOPC | — | 6 | 4 | 15 | — |
| 22 | POPE | Chol | THS | DOPC | 4 | 4 | 1 | 15 |
| 23 | DOPC | CHEMS | — | — | 9 | 1 | — | — |
| 24 | DOPC | CHEMS | — | — | 9 | 1 | — | — |
| 25 | DOPC | CHEMS | — | — | 17 | 3 | — | — |
| 26 | DOPC | CHEMS | — | — | 4 | 1 | — | — |
| 27 | DOPC | CHEMS | — | — | 3 | 1 | — | — |
| 28 | DOPC | CHEMS | — | — | 7 | 3 | — | — |
| 29 | DOPC | THS | — | — | 7 | 3 | — | — |
| 30 | DOPC | DPPG- | — | — | 7 | 3 | — | — |
| | | Na | | | | | | |
| 31 | DOPC | DPPG- | — | — | 9 | 1 | — | — |
| | | Na | | | | | | |
| 32 | POPE | CHEMS | DOPC | — | 6 | 7.5 | 10 | — |
| 33 | POPE | Chol | DOPC | — | 6 | 7.5 | 10 | — |
| 34 | POPE | THS | DOPC | — | 6 | 7.5 | 10 | — |
| 35 | POPC | — | — | — | 1 | — | — | — |
| 36 | POPE | — | — | — | 1 | — | — | — |
| 37 | DOPE | — | — | — | 1 | — | — | — |
| 38 | DLinPC | — | — | — | 1 | — | — | — |
| 39 | POPE | CHEMS | DOPC | — | 4 | 1 | 4 | — |
| 40 | POPE | THS | DOPC | — | 4 | 1 | 4 | — |
| 41 | DOPC | THS | — | — | 9 | 1 | — | — |

Besides liposomes that undergo a phase or conformational transition at acidic pH, enzymatically triggered liposome release approaches can be employed. Enzymatically triggered liposome release has been reviewed by Thompson in Advanced Drug Delivery Reviews 38, pp. 317-338 (1999), the disclosure of which is incorporated by reference herein in its entirety for all purposes. Such enzymatically triggered liposomes described therein can be used in the compositions and methods of the present invention.

In one embodiment, the lipid component of the liposome comprises a sterol. Sterols for use with the invention include, but are not limited to, cholesterol, cholesterol hemi-succinate, esters of cholesterol, salts of cholesterol including cholesterol hydrogen sulfate and cholesterol sulfate, ergosterol, esters of ergosterol including ergosterol hemi-succinate, salts of ergosterol including ergosterol hydrogen sulfate and ergosterol sulfate, lanosterol, esters of lanosterol including lanosterol hemi-succinate, salts of lanosterol including lanosterol hydrogen sulfate, lanosterol sulfate and tocopherols. The tocopherols can include tocopherols, esters of tocopherols including tocopherol hemi-succinates, salts of tocopherols including tocopherol hydrogen sulfates and tocopherol sulfates. The term “sterol compound” includes sterols, tocopherols and the like. A variety of sterols and their water soluble derivatives such as cholesterol hemisuccinate have been used to form liposomes; see, e.g., U.S. Pat. No. 4,721,612 (incorporated by reference herein in its entirety for all purposes). PCT Publication No. WO 85/00968 (incorporated by reference herein in its entirety for all purposes), described a method for reducing the toxicity of drugs by encapsulating them in liposomes comprising alpha-tocopherol and certain derivatives thereof. Also, a variety of tocopherols and their water soluble derivatives have been used to form liposomes, see PCT Publication No. 87/02219, incorporated by reference herein in its entirety for all purposes.

The “antibiotic-to-lipid component” ratio by weight (weight ratios are also referred to herein as “antibiotic-to-lipid”, “antibiotic:lipid” or “antibiotic:lipid component”, abbreviated as “A:L”) in the pharmaceutical composition provided herein, in one embodiment, is about 0.5:1 or greater, about 1:1 or greater (e.g., about 1 (antibiotic):1 (lipid)), about 1.5:1 or greater (e.g., about 1.5 (antibiotic):1 (lipid)), about 2:1 or greater (e.g., about 2 (antibiotic):1 (lipid)), about 2.5:1 or greater (e.g., about 2.5 (antibiotic):1 (lipid).

In another embodiment, the “antibiotic-to-lipid component” ratio by weight is from about 0.5-to-1 (antibiotic-to-lipid component) to about 3-to-1 (antibiotic-to-lipid component), from about 1-to-1 (antibiotic-to-lipid component) to about 3-to-1 (antibiotic-to-lipid component), from about 1.25-to-1 (antibiotic-to-lipid component) to about 3-to-1 (antibiotic-to-lipid component), from about 1.5-to-1 (antibiotic-to-lipid component) to about 3-to-1 (antibiotic-to-lipid component), from about 1.75-to-1 (antibiotic-to-lipid component) to about 3-to-1 (antibiotic-to-lipid component), or from about 2-to-1 (antibiotic-to-lipid component) to about 3-to-1 (antibiotic-to-lipid component).

It should be noted that when an antibiotic-to-lipid ratio is expressed as a range that includes the term “or greater”, the “or greater” refers to the antibiotic component of the ratio. In that regard, the antibiotic-to-lipid component ratio “2-to-1 or greater” includes ratios that have an antibiotic of ≧2 parts by weight.

In another embodiment of the pharmaceutical compositions described herein, a pharmaceutical composition is provided that includes both liposomally encapsulated antibiotic and free antibiotic. The encapsulated antibiotic and free antibiotic can be the same, or different. In one embodiment, both the liposomally encapsulated antibiotic and free antibiotic are aminoglycosides (see Table 1), or pharmaceutically acceptable salts thereof. In a further embodiment, the aminoglycosides, or pharmaceutically acceptable salts thereof, are each amikacin sulfate.

The ratio by weight of free antibiotic to the antibiotic encapsulated liposomes, in one embodiment, is from about 1:100 to about 100:1, from about 1:50 to about 50:1, from about 1:10 to about 10:1, from about 1:5 to about 5:1, from about 1:4 to about 4:1, from about 1:3 to about 3:1, or from about 1:2 to about 2:1.

In order to minimize dose volume and reduce patient dosing time, in one embodiment, it is important that liposomal entrapment of the antibiotic (e.g., the aminoglycoside amikacin or streptomycin, or a pharmaceutically acceptable salt thereof) be highly efficient and that the antibiotic-to-lipid component ratio be as high as possible.

Liposomes described herein are manufactured in one embodiment via a solvent infusion (also referred to as flash precipitation) process. “Solvent infusion” is a process that includes dissolving one or more lipids in a small amount of a process compatible solvent to form a lipid solution and then adding the solution to an aqueous medium containing one or more antibiotics. Typically, a process compatible solvent is one that is miscible with an aqueous solvent and can be washed away in an aqueous process such as dialysis. An alcohol in one embodiment is the solvent employed in the manufacturing process. “Ethanol infusion,” a type of solvent infusion, is a process that includes dissolving one or more lipids in a small amount of ethanol to form a lipid solution and then adding the solution to an aqueous medium containing bioactive agents. The term “solvent infusion” also includes an in-line infusion process where two streams of formulation components are first mixed in-line.

In one embodiment, the liposomal antibiotic formulation of the present invention is prepared by an in-line infusion method where a stream of lipid solution is mixed with a stream of antibiotic solution in-line. For example, the two solutions may be mixed in-line inside a mixing tube preceded by a Y-connector or a T-connector. The in-line infusion method differs from methods where the lipid solution is infused as a stream into a bulk antibiotic solution. The in-line infusion method results in lower lipid to drug ratios (and therefore higher drug-to-lipid ratios) and higher encapsulation efficiencies than a bulk infusion method, where the lipid solution is infused into a bulk antibiotic solution. The in-line infusion process may be further modified by altering parameters such as flow rate, temperature, antibiotic concentration, lipid concentration and salt addition after infusion step. After infusion, one or more wash steps can be employed, e.g., to remove all or substantially all of the unencapsulated antibiotic from the preparation. For example, in some embodiments, unencapsulated antibiotic is removed using tangential flow filtration (TFF) or diafiltration.

In one embodiment, the liposomes described herein are manufactured by one of the methods set forth in U.S. Patent Application Publication No. 2013/0330400 or U.S. Pat. No. 7,718,189, each of which is incorporated by reference in its entirety for all purposes. However, liposomes can be produced by a variety of methods. In one embodiment, one or more of the methods described in U.S. Patent Application Publication No. 2008/0089927 are used herein to produce the liposomes described herein. The disclosure of U.S. Patent Application Publication No. 2008/0089927 is incorporated by reference in its entirety for all purposes. For example, in one embodiment, at least one lipid and an antibiotic are mixed with a coacervate (i.e., a separate liquid phase) to form the liposome composition. The coacervate can be formed to prior to mixing with the lipid, during mixing with the lipid or after mixing with the lipid. Additionally, the coacervate can be a coacervate of the antibiotic.

In one embodiment, the liposomal dispersion is formed by dissolving one or more lipids (i.e., the lipid component of the liposome, or a portion thereof) in an organic solvent forming a lipid solution, and the antibiotic (e.g., aminoglycoside) coacervate forms from mixing an aqueous solution of the antibiotic (e.g., aminoglycoside) with the lipid solution. In a further embodiment, the organic solvent is ethanol.

In one embodiment, liposomes are produced by sonication, extrusion, homogenization, swelling, electroformation, inverted emulsion, interdigitation-fusion or a reverse evaporation method. Bangham's procedure (J. Mol. Biol. (1965)) produces ordinary multilamellar vesicles (MLVs). Lenk et al. (U.S. Pat. Nos. 4,522,803, 5,030,453 and 5,169,637), Fountain et al. (U.S. Pat. No. 4,588,578, incorporated by reference in its entirety for all purposes) and Cullis et al. (U.S. Pat. No. 4,975,282, incorporated by reference in its entirety for all purposes) disclose methods for producing multilamellar liposomes having substantially equal interlamellar solute distribution in each of their aqueous compartments. Paphadjopoulos et al., U.S. Pat. No. 4,235,871, incorporated by reference in its entirety for all purposes, discloses preparation of oligolamellar liposomes by reverse phase evaporation. Each of the methods is amenable for use with the present invention.

Unilamellar vesicles can be produced from MLVs by a number of techniques, for example, the extrusion techniques of U.S. Pat. No. 5,008,050 and U.S. Pat. No. 5,059,421, each of which is incorporated by reference herein in its entirety for all purposes. Sonication and homogenization can be so used to produce smaller unilamellar liposomes from larger liposomes (see, for example, Paphadjopoulos et al. (1968); Deamer and Uster (1983); and Chapman et al. (1968), each of which is incorporated by reference herein in its entirety for all purposes).

The liposome preparation of Bangham et al. (J. Mol. Biol. 13, 1965, pp. 238-252, incorporated by reference herein in its entirety for all purposes) involves suspending phospholipids in an organic solvent which is then evaporated to dryness leaving a phospholipid film on the reaction vessel. Next, an appropriate amount of aqueous phase is added, the mixture is allowed to “swell,” and the resulting liposomes which consist of multilamellar vesicles (MLVs) are dispersed by mechanical means. This preparation provides the basis for the development of the small sonicated unilamellar vesicles described by Papahadjopoulos et al. (Biochim. Biophys. Acta. 135, 1967, pp. 624-638, incorporated by reference herein in its entirety for all purposes), and large unilamellar vesicles.

Techniques for producing large unilamellar vesicles (LUVs), such as, reverse phase evaporation, infusion procedures, and detergent dilution, can be used to produce liposomes for use in the pharmaceutical compositions provided herein. A review of these and other methods for producing liposomes may be found in the text Liposomes, Marc Ostro, ed., Marcel Dekker, Inc., New York, 1983, Chapter 1, which is incorporated herein by reference in its entirety for all purposes. See also Szoka, Jr. et al., (Ann. Rev. Biophys. Bioeng. 9, 1980, p. 467), which is also incorporated herein by reference in its entirety for all purposes.

Other techniques for making liposomes include those that form reverse-phase evaporation vesicles (REV), U.S. Pat. No. 4,235,871, incorporated by reference herein in its entirety for all purposes. Another class of liposomes that may be used is characterized as having substantially equal lamellar solute distribution. This class of liposomes is denominated as stable plurilamellar vesicles (SPLV) as defined in U.S. Pat. No. 4,522,803, and includes monophasic vesicles as described in U.S. Pat. No. 4,588,578, and frozen and thawed multilamellar vesicles (FATMLV) as described above. The disclosure of each of the foregoing references is incorporated by reference in its entirety for all purposes.

The pharmaceutical composition, in one embodiment comprises liposomes with a mean diameter that, if measured by a light scattering method, is of approximately 0.02 microns to approximately 3.0 microns, for example, in the range of from about 0.05 to about 1.0 microns or from about 0.1 microns to about 1.0 microns.

In one aspect of the invention, a method is provided for treating a bacterial infection, or a disease associated with a bacterial infection. The method, in one embodiment, comprises administering to a patient in need thereof, an effective amount of one of the pharmaceutical compositions described herein. As described throughout, the pharmaceutical composition comprises an antibiotic encapsulated in liposomes, and in some embodiments, also comprises free antibiotic (i.e., non-encapsulated). The lipid component or portion thereof of the liposomes, in one embodiment, comprises an unsaturated phospholipid. The liposomal membranes, in one embodiment, undergoes a conformational or phase transition at acidic pH or in response to an environmental factor, for example, an enzymatic reaction. Alternatively, or additionally, the liposome is fusogenic or dynamic, and the lipid component is picked accordingly. Various exemplary liposomal lipid components are provided above at Tables 2, 3 and 4. The bacterial infection in one embodiment is a pulmonary bacterial infection, endothelial infection, brain cell infection or a coronary infection. In a further embodiment, the bacterial infection is a pulmonary bacterial infection. In even a further embodiment, the pulmonary bacterial infection is a pulmonary NTM infection. For example, the pulmonary NTM infection is M. abscessus, M. kansasii, M. fortuitum, M. chelonae, M. xenopi or M. simiae.

The term “treating” includes: (1) preventing or delaying the appearance of clinical symptoms of the state, disorder or condition developing in the subject that may be afflicted with or predisposed to the state, disorder or condition but does not yet experience or display clinical or subclinical symptoms of the state, disorder or condition; (2) inhibiting the state, disorder or condition (i.e., arresting, reducing or delaying the development of the disease, or a relapse thereof in case of maintenance treatment, of at least one clinical or subclinical symptom thereof); and/or (3) relieving the condition (i.e., causing regression of the state, disorder or condition or at least one of its clinical or subclinical symptoms). The benefit to a patient to be treated is either statistically significant or at least perceptible to the patient or to the physician.

“Effective amount” means an amount of an antibiotic (e.g., aminoglycoside or pharmaceutically acceptable salt thereof such as amikacin or amikacin sulfate) used in a composition or method described herein sufficient to result in the desired therapeutic response.

In one embodiment, the method described herein comprises administering a pharmaceutical composition, e.g., a liposomally encapsulated aminoglycoside such as streptomycin or amikacin (e.g., amikacin sulfate) to a patient in need thereof via inhalation, for example, via a nebulizer. In one embodiment, the amount of aminoglycoside provided in the composition is from about 100 mg to about 1000 mg, for example from about 100 mg to about 900 mg, from about 100 mg to about 800 mg, from about 200 mg to about 700 mg, from about 100 mg to about 600 mg. In another embodiment, the amount of aminoglycoside provided in the composition is from about 200 mg to about 800 mg, or from about 300 mg to about 800 mg, or from about 400 mg to about 800 mg.

In one embodiment, the method described herein includes administering one of the pharmaceutical compositions described herein to a patient in need of treatment of a bacterial infection, e.g., a pulmonary NTM infection, for an administration period. The administration period, in one embodiment, includes once daily dosing or twice daily dosing. Dosing of the composition in one embodiment occurs daily (e.g., once a day), every other day, or every third day. Various administration periods can be employed. For example, in one embodiment, an administration period is from about 15 days to about 200 days, e.g., from about 45 days to about 200 days, or from about 45 days to about 170 days, or from about 80 days to about 180 days. For example, the methods provided herein comprise administering to a patient in need thereof an effective amount of one of the compositions described herein once per day in a single dosing session for an administration period of from about 15 days to about 200 days or from about 80 days to about 180 days. In another embodiment, the administration period is from about 50 days to about 90 days.

In one embodiment of the bacterial infection treatment methods described herein, the pharmaceutical composition is administered to a patient in need thereof once per day in a single dosing session. In a further embodiment, the composition is administered as an aerosol via a nebulizer. In another embodiment, the method comprises administering to a patient in need thereof one of the compositions described herein every other day or every three days. In yet another embodiment, the method comprises administering to a patient in need thereof one of a composition described herein twice per day.

In one embodiment, the bacterial infection treatable by the methods and compositions described herein is a mycobacterial infection. The mycobacterial infection in one embodiment is M. tuberculosis or M. leprae. Other embodiments include the treatment of NTM infections such as pulmonary NTM infections, as described throughout.

In one embodiment, a Salmonella (e.g., Salmonella typhimurium, Salmonella typhi), Listeria (e.g., Listeria monocytogens, e.g., Listeria associated with meningitis and spesis) or Francisella bacterial infection, or a disease associated with such an infection is treated with one of the methods and compositions provided herein. In a further embodiment, the method provided herein is used to treat typhoid fever, which is associated with a Salmonella typhi infection.

Francisella is a genus of gram negative bacterium which includes facultative intracellular parasites of macrophages. These bacteria can be targeted with the liposomes provided herein to treat intracellular infections and diseases associated with the respective bacterium. For example, tularemia is treatable by the methods and compositions provided herein, as the infection is caused by Francisella tularensis. F. novicida and F. philmiragia are associated with invasive systemic infections and accordingly can also be targeted with the liposomes provided herein.

In one embodiment, a patient in need of treatment of an Escherichia coli (E. coli) intracellular infection is treated with one of the methods and compositions provided herein. For example, the patient in one embodiment is a bacterial sepsis or meningitis patient. Escherichia coli (E. coli) is known to cause intracellular infections such as bacterial sepsis and meningitis. A patient with one of these infections can be administered one of the pharmaceutical compositions described herein to combat the infection. For example, the pharmaceutical composition can be targeted to brain microvascular endothelial cells to treat neonatal bacterial sepsis and meningitis.

Other pathogens that can be targeted with the liposomes provided herein include, but are not limited to, streptococcal L-forms, Streptobacillus moniliformis, trypanosomes, Coxiella burnetii, trypanosomes, Listeria monocytogens, Streptococcus mutans, P. gingivalis, Eikenella corrodens, Prevotella intermedia, Chlamydia, Tannerella forsythia, Treponema denticola, Mycoplasma, Yersina, Salmonella typhimurium, Borrelia species.

In another embodiment, the bacterial infection is selected from one of the following: Streptobacillus (e.g., Streptobacillus moniliformis), Trypanosoma (e.g., Trypanosoma brucei, associated with sleeping sickness and nagana), Coxiella burnetii (causative agent of Q fever), Streptococcus (e.g., Streptococcal L-forms; S. mutans, S. pyogenes; S. agalactiae); Porphyromonas (e.g., P. gingivalis), Eikenella corrodens, Prevotella (e.g., Prevotella melaninogenica, Prevotella intermedia), Chlamydia (e.g., Chlamydia trachomatis), Tannerella forsythia, Treponema (e.g., Treponema denticola, Treponema palladium, Treponema carateum); Mycoplasma (M. genitalium, M. pneumoniae), Yersina (Y. pestis, Y. aldovae, Y. aleksiciae, Y. bercovien, Y. enterocolitica, Y. entomophaga, Y. frederiksenii, Y. intermdia, Y. kristensenii, Y. massiliensis, Y. mollaretii, Y. nurmii, Y. pekkanenii, Y. philomiragia, Y. pseudotuberculosis, Y. rohdei, Y. ruckeri, Y. similis); Corynebacterium (e.g., C. diphtheria), or a Borrelia infection. Other embodiments include treatment of Rhodococcus (e.g., R. equi and/or R. fascians) infections.

In another embodiment, the bacterial infection is a malaria parasite infection, an Entamoeba (e.g., Entamoeba histolytica, Entamoeba dispar) infection, or a Cryptosporidium (e.g., Cryptosporidium parvum) infection.

Other bacterial infections treatable with the methods and compositions provided herein include but are not limited to Shigellae (e.g., S. boydii, S. dysenteriae, S. flexneri, S. sonnei), L. pneumophila, Rickettsia, a Legionella bacteria such as L. pneumophila, L. longbeachae, L. feeleii, L. micdadei, L. anisa, as well as diseases associated with such pathogens (e.g., Legionnaire's disease and Pontiac fever in the case of Legionella infection; dysentery in the case of Shigella infection; typhus and other arthropod-borne diseases in the case of Rickettsia.

One embodiment of the invention provides a method for treating a Salmonella infection in a patient in need thereof. The method comprises administering to the patient an effective amount of one of the pharmaceutical compositions described herein for an administration period. Another embodiment of the invention provides a method for treating a Listeria infection in a patient in need thereof. The method comprises administering to the patient an effective amount of one of the pharmaceutical compositions described herein for an administration period. Yet another embodiment of the invention provides a method for treating a Mycobacterium infection (e.g., NTM) in a patient in need thereof. The method comprises administering to the patient an effective amount of one of the pharmaceutical compositions described herein for an administration period.

In one embodiment, the bacterial infection is an NTM infection, e.g., a pulmonary NTM infection. Nontuberculous mycobacteria are organisms found in the soil and water that can cause serious lung disease in susceptible individuals, for which there are currently limited effective treatments and no approved therapies. The prevalence of NTM disease is reported to be increasing, and according to reports from the American Thoracic Society is believed to be greater than that of tuberculosis in the U.S. According to the National Center for Biotechnology Information, epidemiological studies show that presence of NTM infection is increasing in developing countries, perhaps because of the implementation of tap water. Women with characteristic phenotype are believed to be at higher risk of acquiring NTM infection along with patients with defects on cystic fibrosis transmembrane conductance regulators. Generally, high risk groups with NTM lung disease for increased morbidity and mortality are those with cavitary lesions, low BMI, advanced age, and a high comorbidity index.

Pulmonary NTM infection (also referred to herein as NTM lung infection) is often a chronic condition that can lead to progressive inflammation and lung damage, and is characterized by bronchiectasis and cavitary disease. NTM infections often require lengthy hospital stays for medical management. Treatment usually involves multi-drug regimens that can be poorly tolerated and have limited effectiveness, especially in patients with severe disease or in those who have failed prior treatment attempts. According to a company-sponsored patient chart study conducted by Clarity Pharma Research, approximately 50,000 patients suffering from NTM lung disease visited physician offices in the U.S. during 2011.

Management of pulmonary disease caused by NTM infection includes lengthy multidrug regimens, which are often associated with drug toxicity and suboptimal outcomes. Achieving NTM culture negativity is one of the objectives of treatment and represents the most clinically important microbiologic endpoint in patients with pulmonary NTM infection.

In one embodiment, the present invention provides a method for treating a pulmonary NTM infection in a patient in need thereof. The method in one embodiment comprises administering to the patient, an effective amount of one of the pharmaceutical compositions described herein (see, e.g., Tables 1, 2, 3 and 4 for lipid and antibiotic components of such compositions). The antibiotic, in one embodiment, is an aminoglycoside, or a pharmaceutically acceptable salt thereof. In one embodiment, the NTM lung infection is a recalcitrant nontuberculous mycobacterial lung infection. The patient, in one embodiment, exhibits an increased number of meters walked in the 6 minute walk test (6MWT), as compared to prior to treatment and/or an NTM culture conversion to negative, during the administration period or after the administration period. Culture conversion is defined as at least three consecutive monthly sputum samples that test negative for NTM bacteria. Testing for culture conversion can begin during the administration period.

The therapeutic response can be any response that a user (e.g., a clinician) will recognize as an effective response to the therapy. For NTM infections, the therapeutic response will generally be a reduction, inhibition, delay or prevention in growth of or reproduction of one or more NTM, or the killing of one or more NTM. A therapeutic response may also be reflected in an improvement in pulmonary function, for example forced expiratory volume in one second (FEV₁). In one embodiment, where a patient is treated for an NTM lung infection, the therapeutic response is measured as the change from baseline on the full semi quantitative scale for mycobacterial culture or an improvement in the distance walked in the 6MWT. It is further within the skill of one of ordinary skill in the art to determine appropriate treatment duration, appropriate doses, and any potential combination treatments, based upon an evaluation of therapeutic response.

The NTM lung infection treatable by the methods and compositions described herein, in one embodiment, is M. avium, M. avium subsp. hominissuis (MAH), M. avium subsp. paratuberculosis (Crohn's disease), M. abscessus, M. chelonae, M. indicus pranii, M. bolletii, M. kansasii, M. ulcerans, M. avium, M. avium complex (MAC) (M. avium and M. intracellulare), M. conspicuum, M. peregrinum, M. immunogenum, M. xenopi, M. massiliense, M. marinum, M. malmoense, M. mucogenicum, M. nonchromogenicum, M. scrofulaceum, M. simiae, M. smegmatis, M. szulgai, M. terrae, M. terrae complex, M. haemophilum, M. genavense, M. asiaticum, M. shimoidei, M. gordonae, M. nonchromogenicum, M. triplex, M. lentiflavum, M. celatum, M. fortuitum, M. fortuitum complex (M. fortuitum and M. chelonae) or a combination thereof. In a further embodiment, the nontuberculous mycobacterial lung infection is M. avium complex (MAC) (M. avium and M. intracellulare), M. abscessus or M. avium. In a further embodiment, the M. avium infection is M. avium subsp. hominissuis. In one embodiment, the nontuberculous mycobacterial lung infection is M. avium complex (MAC) (M. avium and M. intracellulare). In another embodiment, the NTM lung infection is a recalcitrant nontuberculous mycobacterial lung infection. Other embodiments include the treatment of intracellular pulmonary NTM infections M. abscessus, M. kansasii, M. fortuitum, M. chelonae, M. xenopi or M. simiae, or a combination thereof.

As described throughout, the compositions and systems described herein are used to treat an infection caused by a nontuberculous mycobacterium (NTM). In one embodiment, the compositions and systems described herein are used to treat an infection caused by Mycobacterium abscessus or Mycobacterium avium. In even a further embodiment, the Mycobacterium avium infection is Mycobacterium avium subsp. hominissuis.

In one embodiment, a patient is treated for a Mycobacterium abscessus, M. kansasii, M. abscessus, M. fortuitum, Mycobacterium avium or a M. avium complex (M. avium-intracellulare, abbreviated “MAC”) lung infection via inhalation delivery of a liposomal aminoglycoside composition. In a further embodiment, the aminoglycoside is amikacin sulfate and is administered once per day for a single dosing session. In even a further embodiment, the NTM lung infection is MAC.

The NTM lung infection, in one embodiment, is associated with cavitary lesions. In one embodiment, the NTM lung infection is a nodular infection. In a further embodiment, the NTM lung infection is a nodular infection with minimal cavitary lesions.

The present invention provides in one aspect, a method for treating or providing prophylaxis against a pulmonary NTM infection. Treatment is achieved via delivery of a pharmaceutical composition comprising a liposomal aminoglycoside composition by inhalation via nebulization of the composition. In one embodiment, the composition comprises an aminoglycoside encapsulated in liposomes, e.g., an aminoglycoside selected from one or more of the aminoglycosides of Tables 1, or a pharmaceutically acceptable salt thereof. Pharmaceutical compositions described herein can also include free antibiotic, as described above.

In one embodiment, the compositions provided herein are tested for their ability to treat NTM infections in in vivo and in vitro infection models, for example, the infection models described in U.S. Patent Application Publication No. 2015/0283133, incorporated by reference herein in its entirety for all purposes.

The macrophage test system described in, or adapted from Rose (see Examples) can be employed (Rose et al. (2014). PLoS One 9(9), e108703. doi:10.1371/journal.pone.0108703, incorporated by reference herein in its entirety for all purposes). The source of macrophages in some embodiments is the THP-1 human monocyte cell line (ATCC) and can be cultured, e.g., in RPMI-1640 medium (Gibco, Chicago, Ill.) supplemented with 5% fetal bovine serum (Gemini, Sacramento, Calif.) and 2 mM of L-glutamine. THP-1 cells as described in Rose are maintained at 37° C. in an atmosphere of 5% CO². Monocytes are then grown to 5×10⁶ cells per mL, washed and resuspended to a concentration of 1×10⁶ cells per mL and seeded. Monolayers are then treated with 0.5 mg of phorbol myristate acetate per ml for 24 hours to stimulate the maturation of the monocytes. Bacteria are prepared for infection by resuspension in Hank's buffered salt solution (HBSS) to concentrations of 3×10⁸ CFU/ml by comparison with a McFarland #1 turbidity standard. Prior to the infection of macrophage monolayers, the suspension is agitated and passed through a 23-gauge needle ten times to disperse clumps. Suspensions are serially diluted and plated onto 7H10 agar to confirm the concentration of the inoculum. The monolayers are infected with NTM, e.g., M. avium subsp. hominissuis (MAH), M. avium, M. ab. or other NTM species mentioned herein, at a multiplicity of infection of 10:1. After 1 hour of infection, the extracellular bacteria are removed via washing, and the intracellular infection is allowed to incubate for 24 hours. Following the establishment of the infection baseline, the addition of liposomal antibiotic or controls is performed for the desired time course, e.g., once daily for 4 days. Lysis of THP-1 cells is carried out with a 10 min. incubation in 0.1% Triton X-100 in sterile H₂O followed by mixing, diluting, and plating onto 7H10 agar plates for CFU enumeration.

In another embodiment, THP-1 cells can be seeded in PMA and cultured for 48 hours, exposed to M. abscessus (type strain 19977 or clinical isolate Stanford A or NIH26) at multiplicity of infection (MOI) of approx. 2 for 1 hour followed by incubation with 50 mg/mL antibiotic for 23 hours to reduce the extracellular NTM population. Liposomal antibiotic formulations can be added at various concentrations and cells incubated for 24 hours at 37° C. At the end of a user chosen 24 hour treatment cycles, cells are washed, lysed, serially diluted, plated, and incubated at 37° C. for 4 days to determine final intracellular CFU counts.

In one embodiment, a patient subjected to one of the NTM methods described herein exhibits an NTM culture conversion to negative during the administration period of the liposomal aminoglycoside composition, or after the administration period has concluded. Culture conversion is defined in one embodiment as at least three consecutive monthly sputum samples that test negative for NTM bacteria. Testing for culture conversion can begin during the administration period. The time to conversion, in one embodiment, is about 10 days, or about 20 days or about 30 days or about 40 days, or about 50 days, or about 60 days, or about 70 days, or about 80 days, or about 90 days, or about 100 days or about 110 days. In another embodiment, the time to conversion is from about 20 days to about 200 days, from about 20 days to about 190 days, from about 20 days to about 180 days, from about 20 days to about 160 days, from about 20 days to about 150 days, from about 20 days to about 140 days, from about 20 days to about 130 days, from about 20 days to about 120 days, from about 20 days to about 110 days, from about 30 days to about 110 days, or from about 30 days to about 100 days after the method has begun.

In some embodiments, the patient experiences an improvement in lung function for at least 15 days after the administration period ends, as compared to the FEV₁ of the patient prior to treatment. For example, the patient may experience an increase in FEV₁, an increase in blood oxygen saturation, or both. In some embodiments, the patient has an FEV₁ (after the administration period or treatment cycle) that is increased by at least 5% over the FEV₁ prior to the administration period. In other embodiments, FEV₁ is increased by 5 to 50% over the FEV₁ prior to the administration period. In other embodiments, FEV₁ is increased by 25 to 500 mL over FEV₁ prior to the administration period. In some embodiments, blood oxygen saturation is increased by at least 1% over oxygen saturation prior to the administration period.

In one embodiment, the 6MWT is used to assess the effectiveness of the treatment methods provided herein. The 6MWT is used for the objective evaluation of functional exercise capacity and is a practical, simple test that measures the distance that a patient can walk in a period of 6 minutes (see American Thoracic Society. (2002). Am J Respir Crit Care Med. 166, pp. 111-117, incorporated by reference herein in its entirety for all purposes).

In one embodiment, a patient subjected to one of the NTM methods described herein exhibits an increased number of meters walked in the 6MWT, as compared to prior to undergoing the treatment method. The increased number of meters walked in the 6MWT, in one embodiment, is about 5 meters, about 10 meters, about 15 meters, about 20 meters, about 25 meters, about 30 meters, about 35 meters, about 40 meters, about 45 meters, or about 50 meters. In another embodiment, the increased number of meters walked in the 6MWT is at least about 5 meters, at least about 10 meters, at least about 15 meters, at least about 20 meters, at least about 25 meters, at least about 30 meters, at least about 35 meters, at least about 40 meters, at least about 45 meters, or at least about 50 meters. In yet another embodiment, the increased number of meters walked in the 6MWT is from about 5 meters to about 50 meters, or from about 5 meters to about 40 meters, or from about 5 meters to about 30 meters or from about 5 meters to about 25 meters.

In another embodiment, a patient subjected to one of the NTM methods described herein exhibits a greater number of meters walked in the 6MWT, as compared to a patient undergoing a non-liposomal aminoglycoside treatment. The greater number of meters walked in the 6MWT, as compared to a patient undergoing a non-liposomal aminoglycoside treatment, in one embodiment, is about 5 meters, about 10 meters, about 15 meters, about 20 meters, about 25 meters, about 30 meters, about 35 meters, about 40 meters, about 45 meters, about 50 meters, about 60 meters, about 70 meters or about 80 meters. In another embodiment, the greater number of meters walked in the 6MWT is at least about 5 meters, at least about 10 meters, at least about 15 meters, at least about 20 meters, at least about 25 meters, at least about 30 meters, at least about 35 meters, at least about 40 meters, at least about 45 meters, or at least about 50 meters. In yet another embodiment, the greater number of meters walked in the 6MWT is from about 5 meters to about 80 meters, or from about 5 meters to about 70 meters, or from about 5 meters to about 60 meters or from about 5 meters to about 50 meters.

Suitable delivery routes of the compositions provided herein will be apparent to one of ordinary skill in the art depending on the intracellular bacterial infection to be treated. For example, in the case of a pulmonary infection, inhalation administration can be employed. Other suitable routes of administration for the pharmaceutical compositions provided herein include, but are not limited to, parental administration (e.g., intramuscular, intravenous, intranasal, intraperitoneally, intraarterial, intrathecal, subcutaneous).

In one embodiment, administration of a composition provided herein is intravenous administration.

In one embodiment, administration of a composition provided herein is intramuscular administration.

In another embodiment, administration of the composition provided herein is subcutaneous administration.

As provided herein, the methods described herein in one embodiment comprise administering to a patient in need of treatment of a bacterial lung infection (e.g., an NTM lung infection), an effective amount of one of the pharmaceutical compositions described herein via inhalation. Various inhalation delivery devices can be employed in the methods of treatments described herein. For example, the inhalation delivery device can be a nebulizer or a dry powder inhaler. The device can contain and be used to deliver a single dose of the pharmaceutical composition or the device can contain and be used to deliver multi-doses of the composition of the present invention.

In one embodiment, inhalation delivery is conducted via a nebulizer. The nebulizer provides an aerosol mist of the composition for delivery to the lungs of the patient. A “nebulizer” or an “aerosol generator” is a device that converts a liquid into an aerosol of a size that can be inhaled into the respiratory tract. Pneumonic, ultrasonic, electronic nebulizers, e.g., passive electronic mesh nebulizers, active electronic mesh nebulizers and vibrating mesh nebulizers are amenable for use with the invention if the particular nebulizer emits an aerosol with the required properties, and at the required output rate.

The process of pneumatically converting a bulk liquid into small droplets is called atomization. The operation of a pneumatic nebulizer requires a pressurized gas supply as the driving force for liquid atomization. Ultrasonic nebulizers use electricity introduced by a piezoelectric element in the liquid reservoir to convert a liquid into respirable droplets. Various types of nebulizers are described in Respiratory Care, Vol. 45, No. 6, pp. 609-622 (2000), the disclosure of which is incorporated herein by reference in its entirety. The terms “nebulizer” and “aerosol generator” are used interchangeably throughout the specification.

In one embodiment, the system provided herein comprises a nebulizer selected from an electronic mesh nebulizer, pneumonic (jet) nebulizer, ultrasonic nebulizer, breath-enhanced nebulizer and breath-actuated nebulizer. In one embodiment, the nebulizer is portable.

In one embodiment, the method for treating a pulmonary intracellular bacterial infection such as an NTM infection is carried out via administration of a liposomal aminoglycoside composition to a patient in need thereof via a nebulizer in once daily dosing sessions. In a further embodiment, the aminoglycoside is amikacin, e.g., amikacin sulfate. In even a further embodiment, the nebulizer is one of the nebulizers described in U.S. Patent Application Publication No. 2013/0330400, incorporated by reference herein in its entirety for all purposes.

The principle of operation of a pneumonic nebulizer is generally known to those of ordinary skill in the art and is described, e.g., in Respiratory Care, Vol. 45, No. 6, pp. 609-622 (2000), the disclosure of which is incorporated herein by reference in its entirety. Briefly, a pressurized gas supply is used as the driving force for liquid atomization in a pneumatic nebulizer. Compressed gas is delivered, which causes a region of negative pressure. The solution to be aerosolized is then delivered into the gas stream and is sheared into a liquid film. This film is unstable and breaks into droplets because of surface tension forces. Smaller particles, i.e., particles with the mass median aerodynamic diameter (MMAD) and fine particle fraction (FPF) properties described herein, can then be formed by placing a baffle in the aerosol stream. In one pneumonic nebulizer embodiment, gas and solution is mixed prior to leaving the exit port (nozzle) and interacting with the baffle. In another embodiment, mixing does not take place until the liquid and gas leave the exit port (nozzle). In one embodiment, the gas is air, 02 and/or CO₂.

“Mass median aerodynamic diameter” or “MMAD” is normalized regarding the aerodynamic separation of aqua aerosol droplets and is determined impactor measurements, e.g., the Andersen Cascade Impactor (ACI) or the Next Generation Impactor (NGI). The gas flow rate, in one embodiment, is 28 Liter per minute by the Andersen Cascade Impactor (ACI) and 15 Liter per minute by the Next Generation Impactor (NGI). “Geometric standard deviation” or “GSD” is a measure of the spread of an aerodynamic particle size distribution. “Fine particle fraction” or “FPF,” as used herein, refers to the fraction of the aerosol having a particle size less than 5 μm in diameter, as measured by cascade impaction. FPF is usually expressed as a percentage.

In one embodiment, droplet size and output rate can be tailored in a pneumonic nebulizer. However, consideration should be paid to the composition being nebulized, and whether the properties of the composition (e.g., % associated aminoglycoside) are altered due to the modification of the nebulizer. For example, in one embodiment, the gas velocity and/or pharmaceutical composition velocity is modified to achieve the output rate and droplet sizes of the present invention. Additionally or alternatively, the flow rate of the gas and/or solution can be tailored to achieve the droplet size and output rate of the invention. For example, an increase in gas velocity, in one embodiment, decreased droplet size. In one embodiment, the ratio of pharmaceutical composition flow to gas flow is tailored to achieve the droplet size and output rate of the invention. In one embodiment, an increase in the ratio of liquid to gas flow increases particle size.

In one embodiment, a pneumonic nebulizer output rate is increased by increasing the fill volume in the liquid reservoir. Without wishing to be bound by theory, the increase in output rate may be due to a reduction of dead volume in the nebulizer. Nebulization time, in one embodiment, is reduced by increasing the flow to power the nebulizer. See, e.g., Clay et al. (1983). Lancet 2, pp. 592-594 and Hess et al. (1996). Chest 110, pp. 498-505; each of which is incorporated by reference herein in its entirety.

In one embodiment, a reservoir bag is used to capture aerosol during the nebulization process, and the aerosol is subsequently provided to the subject via inhalation. In another embodiment, the nebulizer provided herein includes a valved open-vent design. In this embodiment, when the patient inhales through the nebulizer, nebulizer output is increased. During the expiratory phase, a one-way valve diverts patient flow away from the nebulizer chamber.

In one embodiment, the nebulizer provided herein is a continuous nebulizer. In other words, refilling the nebulizer with the pharmaceutical composition while administering a dose is not needed.

In one embodiment, the nebulizer provided herein does not use an air compressor and therefore does not generate an air flow. In one embodiment, aerosol is produced by the aerosol head which enters the mixing chamber of the device. When the patient inhales, air enters the mixing chamber via one-way inhalation valves in the back of the mixing chamber and carries the aerosol through the mouthpiece to the patient. On exhalation, the patient's breath flows through the one-way exhalation valve on the mouthpiece of the device. In one embodiment, the nebulizer continues to generate aerosol into the mixing chamber which is then drawn in by the subject on the next breath—and this cycle continues until the nebulizer medication reservoir is empty.

In one embodiment, the nebulization time of an effective amount of a pharmaceutical composition provided herein is less than 20 minutes, less than 18 minutes, less than 16 minutes, less than 15 minutes, less than 10 minutes or less than 5 minutes. In one embodiment, the nebulization time of an effective amount of an aminoglycoside composition provided herein is less than 15 minutes or less than 13 minutes. In one embodiment, the nebulization time of an effective amount of a pharmaceutical composition provided herein is about 13 minutes. In yet another embodiment, the nebulization time of a pharmaceutical composition is from about 1 minute to about 15 minutes, from about 1 minute to about 14 minutes, from about 1 minute to about 13 minutes, from about 1 minute to about 12 minutes, from about 1 minute to about 11 minutes, from about 1 minute to about 10 minutes, from about 1 minute to about 9 minutes, from about 1 minute to about 8 minutes, from about 1 minute to about 7 minutes or from about 1 minute to about 6 minutes. In even another embodiment, the nebulization time of a pharmaceutical composition is from about 1 minute to about 15 minutes, from about 2 minutes to about 15 minutes, from about 3 minutes to about 15 minutes, from about 4 minutes to about 15 minutes, from about 5 minutes to about 15 minutes, from about 6 minutes to about 15 minutes, from about 7 minutes to about 15 minutes, from about 8 minutes to about 15 minutes, from about 9 minutes to about 15 minutes or from about 10 minutes to about 15 minutes,

In one embodiment, the composition described herein is administered once daily to a patient in need thereof, during the administration period.

In one embodiment, prior to nebulization of the antibiotic (e.g., aminoglycoside) composition, about 10% to about 100% of the antibiotic (e.g., aminoglycoside) present in the composition is liposomally encapsulated. In a further embodiment, the antibiotic is an aminoglycoside. In even a further embodiment, the aminoglycoside is amikacin.

In another embodiment, prior to nebulization, about 30% to about 90%, about 40% to about 90%, or about 50% to about 90%, or about 60% to about 90% of the antibiotic present in the composition is liposomally encapsulated. In another embodiment, prior to nebulization, about about 30% to about 99%, 40% to about 99%, about 50% to about 99%, about 60% to about 99%, or about 70% to about 99%, or about 80% to about 99% or about 90% to about 99% or about 95% to about 99% of the antibiotic present in the composition is liposomally encapsulated. In a further embodiment, the aminoglycoside is amikacin or tobramycin, or a pharmaceutically acceptable salt thereof. In even a further embodiment, the aminoglycoside is amikacin. In another embodiment, prior to nebulization, about 98% of the aminoglycoside present in the composition is liposomally encapsulated. In a further embodiment, the aminoglycoside is amikacin or tobramycin. In even a further embodiment, the aminoglycoside is amikacin (e.g., as amikacin sulfate).

In one embodiment, upon nebulization, about 5% to about 90% of the liposomally encapsulated antibiotic is released, due to shear stress on the liposomes. In a further embodiment, the antibiotic is an aminoglycoside such as amikacin. In another embodiment, upon nebulization, about 10% to about 50%, or about 20% to about 40% of the liposomally encapsulated antibiotic is released from the liposomes, due to shear stress on the liposomes.

In one embodiment, the percent associated antibiotic post-nebulization is measured by reclaiming the aerosol from the air by condensation in a cold-trap, and the liquid is subsequently assayed for free and encapsulated aminoglycoside.

Besides a nebulizer, a non-nebulizer type inhalation delivery device such as a dry powder inhaler (DPI) is used to deliver one of the pharmaceutical compositions described herein. In one embodiment, the DPI generates particles having an MMAD of from about 1 μm to about 10 μm, or about 1 μm to about 9 μm, or about 1 μm to about 8 μm, or about 1 μm to about 7 μm, or about 1 μm to about 6 μm, or about 1 μm to about 5 μm, or about 1 μm to about 4 μm, or about 1 μm to about 3 μm, or about 1 μm to about 2 μm in diameter, as measured by the NGI or ACI. In another embodiment, the DPI generates a particles having an MMAD of from about 1 μm to about 10 μm, or about 2 μm to about 10 μm, or about 3 μm to about 10 μm, or about 4 μm to about 10 μm, or about 5 μm to about 10 μm, or about 6 μm to about 10 μm, or about 7 μm to about 10 μm, or about 8 μm to about 10 μm, or about 9 μm to about 10 μm, as measured by the NGI or ACI.

In another embodiment, the methods provided herein are implemented for the treatment or prophylaxis of one or more pulmonary NTM infections in a cystic fibrosis (CF) patient.

In one embodiment, the patient in need of treatment of the pulmonary NTM infection is a bronchiectasis patient. In one embodiment, the bronchiectasis is non-CF bronchiectasis. In another embodiment, the bronchiectasis is associated with CF in a patient in need of treatment.

In another embodiment, the patient in need of treatment of the pulmonary NTM infection is a COPD patient. In yet another embodiment, the patient in need of treatment of the pulmonary NTM infection is an asthma patient.

In one embodiment, a patient in need of treatment with one of the methods described herein is a CF patient, a bronchiectasis patient, a ciliary dyskinesia patient, a chronic smoker, a chronic obstructive pulmonary disorder (COPD) patient, or a patient who has been previously non-responsive to treatment. In another embodiment, a cystic fibrosis patient is treated for a pulmonary NTM infection with one of the methods provided herein. In yet another embodiment, the patient is a bronchiectasis patient, a COPD patient or an asthma patient. The pulmonary NTM infection, in one embodiment, is MAC, M. kansasii, M. abscessus, or M. fortuitum. In a further embodiment, the pulmonary NTM infection is a MAC infection.

A patient subjected to the methods described herein, in one embodiment, has a co-morbid condition. For example, in one embodiment, the patient in need of treatment with one of the methods described herein has diabetes, mitral valve disorder (e.g., mitral valve prolapse), acute bronchitis, pulmonary hypertension, pneumonia, asthma, trachea cancer, bronchus cancer, lung cancer, cystic fibrosis, pulmonary fibrosis, a larynx anomaly, a trachea anomaly, a bronchus anomaly, aspergillosis, HIV or bronchiectasis, in addition to the pulmonary NTM infection.

In one embodiment, the pharmaceutical composition provided herein is administered to a patient in need of treatment of a bacterial infection with one or more additional therapeutic agents. The one or more additional therapeutics agents in one embodiment, is administered orally. In another embodiment, the one or more additional therapeutics agents in one embodiment, is administered intravenously. In yet another embodiment, the one or more additional therapeutics agents in one embodiment, is administered via inhalation.

The one or more additional therapeutic agents in one embodiment, is a macrolide antibiotic. In a further embodiment, the macrolide antibiotic is azithromycin, clarithromycin, erythromycin, carbomycin A, josamycin, kitamycin, midecamycin, oleandomycin, solithromycin, spiramycin, troleandomycin, tylosin, roxithromycin, or a combination thereof. In a further embodiment, the macrolide antibiotic is administered orally.

In one embodiment, the one or more additional therapeutic agents is the macrolide antibiotic azithromycin, clarithromycin, erythromycin, or a combination thereof. In a further embodiment, the macrolide antibiotic is administered orally.

In another embodiment, the liposomal aminoglycoside composition provided herein is administered to a patient in need of treatment of an NTM lung disease with one or more additional therapeutic agents, and the one or more additional therapeutic agents is a rifamycin compound. In a further embodiment, the rifamycin is rifampin. In another embodiment, the rifamycin is rifabutin, rifapentine, rifaximin, or a combination thereof.

In yet embodiment, the one or more additional therapeutic agents is a quinolone. In a further embodiment, the quinolone is a fluoroquinolone. In another embodiment, the quinolone is ciprofloxacin, levofloxacin, gatifloxacin, enoxacin, levofloxacin, ofloxacin, moxifloxacin, trovafloxacin, or a combination thereof.

In one embodiment, a second therapeutic agent is administered to the patient in need of NTM treatment, and the second therapeutic agent is a second aminoglycoside. In a further embodiment, the second aminoglycoside is amikacin, apramycin, arbekacin, astromicin, bekanamycin, boholmycin, brulamycin, capreomycin, dibekacin, dactimicin, etimicin, framycetin, gentamicin, H107, hygromycin, hygromycin B, inosamycin, K-4619, isepamicin, KA-5685, kanamycin, neomycin, netilmicin, paromomycin, plazomicin, ribostamycin, sisomicin, rhodestreptomycin, sorbistin, spectinomycin, sporaricin, streptomycin, tobramycin, verdamicin, vertilmicin, a pharmaceutically acceptable salt thereof, or a combination thereof. In a further embodiment, the second aminoglycoside is administered intravenously or via inhalation. In one embodiment the second aminoglycoside is streptomycin.

In another embodiment, the liposomal aminoglycoside composition provided herein is administered to a patient in need of treatment of an NTM lung disease with one or more additional therapeutic agents, and the one or more additional therapeutic agents is ethambutol, isoniazid, cefoxitin or imipenem.

### Examples

The present invention is further illustrated by reference to the following Examples. However, it should be noted that this Example, like the embodiments described above, are illustrative and are not to be construed as restricting the scope of the invention in any way.

### Example 1: Design of pH Sensitive Liposomal Compositions

The following four liposome compositions were used (molar ratio of lipid components provided in parentheses). Calcein was encapsulated into liposomes via a flash precipitation method.

Dipalmitoylphosphatidylcholine (DPPC)/cholesterol (50/50) (Composition 1)

### DOPE/CHEMS (6/4) (Composition 2);

### POPE/Chol/THS (4/4/1) (Composition 3);

### NAPE/DODAP/DOPC (4/4/2) (Composition 4).

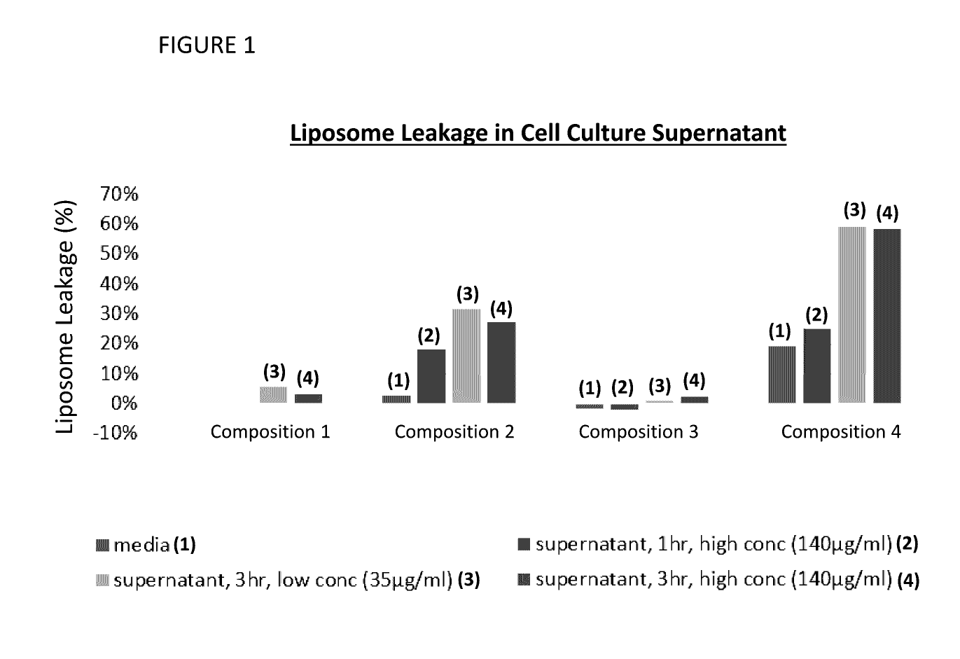

The liposomal compositions were tested in a cell culture supernatant assay to determine the amount of calcein leakage as a function of various incubation times and concentrations of calcein. Media used was 2% FBS. Results of this assay are provided in FIG. 1. Composition 3 did not exhibit leakage in blank 2% FBS media or supernatant, even after 3 hr. cell culture. Composition 2 did not exhibit leakage in 2% FBS media but gradually leaked up to 30% after 3 hr. cell culture. Composition 4 exhibited 20% leakage in blank 2% FBS media and leaked up to 60% after 3 hr. cell culture.

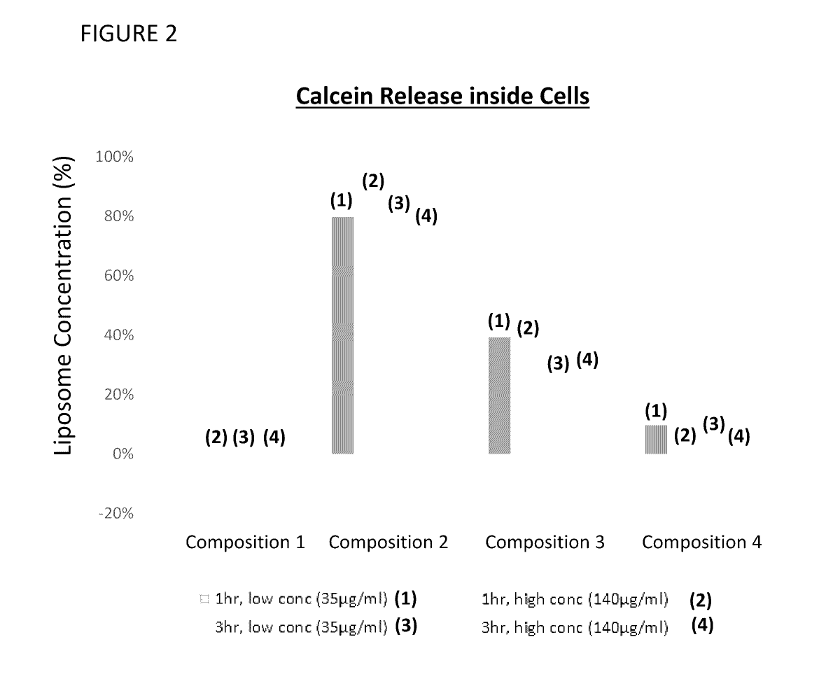

The same compositions were tested for their calcein release efficiency. Release efficiency is a measure of intracellular calcein release, after accounting for the leakage in the extracellular environment. The results of this experiment are provided in FIG. 2. Composition 2 was found to have the highest “releasing efficiency”. Composition 3 exhibited less release efficiency, as compared to Composition 2, and Composition 4 exhibited the least release efficiency. The release efficiency of Composition 1 was found to be absent.

Finally, uptake efficiency of the four compositions was tested in macrophage cells. Composition 1 liposome was added into cell culture with 20% v/v sugar solution to avoid aggregation. It was found that among Compositions 2, 3 and 4, Compositions 2 and 4 have the highest uptake efficiency, while Composition 3 (POPE) had about half the uptake efficiency as Composition 4.

### Example 2: In Vitro Characterization of Liposomal Amikacin Formulations

The following formulations were evaluated for various parameters such as particle size, and amikacin sulfate-to-lipid ratio as shown in Table 5. Formulations were manufactured via an in-line infusion process. The lipid stream was infused at a lipid concentration of 20 mg/mL to 80 mg/mL at a flow rate of 10 mL/min to about 25 mL/min, and the aqueous amikacin sulfate stream was infused at an amikacin sulfate concentration of 40 mg/mL to about 100 mg/mL at a flow rate of 15 mL/min to 40 mL/min. After infusion, the products were washed to remove unencapsulated amikacin sulfate using tangential flow filtration (TFF).

TABLE 5
Summary of Liposomal Amikacin Formulations

| | | | | | | | | | Amikacin | | |
|---|---|---|---|---|---|---|---|---|---|---|---|
| | Component (Comp.) Identity | | | | Target Molar Ratio | | | | sulfate- | Particle Size | |
| | Comp. | Comp. | Comp. | Comp. | Comp. | Comp. | Comp. | Comp. | to-lipid | (postTFF) | |
| # | 1 | 2 | 3 | 4 | 1 | 2 | 3 | 4 | ratio | Size (nm) | % PD |
| 3 | DOPE | CHEMS | | | 3 | 2 | | | — | 220 | 57 |
| 4 | POPE | Chol | THS | | 4 | 4 | 1 | | 0.52 | 205 | 24 |
| 5 | NAPE | DODAP | DOPC | | 2 | 2 | 1 | | 0.44 | 223 | 36 |
| 6 | POPC | CHEMS | OAlc | | 5 | 5 | 8 | | 0.3 | 207 | 25 |
| 7 | diolein | CHEMS | — | | 3 | 2 | | | 0.86 | 211 | 23 |
| 8 | DOPC | — | — | — | 1 | — | — | — | — | 137 | 21 |
| 9 | DOPE | Oleic Acid | — | — | 7 | 3 | — | — | — | — | — |
| 10 | DOPE | DOPC | — | — | 3 | 2 | — | — | 1.33 | 85 | 24 |
| 11 | Chol | DOPC | DPPS | — | 5 | 4 | 1 | — | — | — | — |
| 12 | DOPE | CHEMS | DOPC | — | 6 | 4 | 2 5 | — | 0.68 | 156 | 57 |
| 13 | DOPE | CHEMS | DOPC | — | 4.5 | 3 | 5 | — | — | — | — |
| 14 | POPE | Chol | THS | DOPC | 4 | 4 | 1 | 2.5 | 0.51 | 284 | 18 |
| 15 | POPE | Chol | THS | DOPC | 6 | 6 | 1.5 | 10 | 2.11 | 268 | 33 |
| 16 | POPE | Chol | THS | POPC | 4 | 4 | 1 | 2.5 | 0.77 | 204 | 57 |
| 18 | DOPE | CHEMS | POPC | — | 6 | 4 | 2.5 | — | 0.88 | 102 | 24 |
| 19 | DOPE | CHEMS | POPC | — | 4.5 | 3 | 5 | — | 0.54 | 116 | 12 |
| 20 | DPPC | DPPG | — | — | 19 | 1 | — | — | — | — | — |
| 21 | DOPE | CHEMS | DOPC | — | 3 | 2 | 7.5 | | 0.63 | 140 | 42 |
| 22 | POPE | Chol | THS | DOPC | 4 | 4 | 1 | 15 | 0.50 | 223 | 31 |
| 23 | DOPC | CHEMS | — | — | 19 | 1 | — | — | 0.30 | 561 | 13 |
| 24 | DOPC | CHEMS | — | — | 9 | 1 | — | — | 0.52 | 145 | 48 |
| 25 | DOPC | CHEMS | — | — | 17 | 3 | — | — | — | — | — |
| 26 | DOPC | CHEMS | — | — | 4 | 1 | — | — | 0.39 | 146 | 37 |
| 27 | DOPC | CHEMS | — | — | 3 | 1 | — | — | 0.25 | 103 | 18 |
| 28 | DOPC | CHEMS | — | — | 7 | 3 | — | — | 0.35 | 91 | 11 |
| 29 | DOPC | THS | — | — | 7 | 3 | — | — | 0.39 | 108 | 12 |
| 30 | DOPC | DPPG-Na | — | — | 7 | 3 | — | — | 0.78 | 160 | 23 |
| 31 | DOPC | DPPG-Na | — | — | 9 | 1 | — | — | — | 147 | 23 |
| 32 | POPE | CHEMS | DOPC | — | 6 | 7.5 | 10 | — | — | 152 | 23 |
| 33 | POPE | Chol | DOPC | — | 6 | 7.5 | 10 | — | 2.02 | 161 | 24 |
| 34 | POPE | THS | DOPC | — | 6 | 7.5 | 10 | — | 0.4 | 104 | 22 |
| 35 | POPC | — | — | — | 1 | — | — | — | 0.19 | 136 | 23 |
| 36 | POPE | — | — | — | 1 | — | — | — | 2.32 | 102 | 21 |
| 37 | DOPE | — | — | — | 1 | — | — | — | 0.31 | 385 | 14 |
| 38 | DLinPC | — | — | — | 1 | — | — | — | 0.46 | 122 | 21 |
| 39 | POPE | CHEMS | DOPC | — | 4 | 1 | 4 | — | 2.5 | — | — |
| 40 | POPE | THS | DOPC | — | 4 | 1 | 4 | — | — | — | — |
| 41 | DOPC | THS | — | — | 9 | 1 | — | — | — | — | — |

POPE: 1-palmitoyl-2-oleoyl-sn-glycero-3-phosphoethanolamine;
Chol: cholesterol;
DOPC: 1,2-dioleoyl-sn-glycero-3-phosphocholine;
CHEMS: cholesterol hemi-succinate;
THS: tocopherol hemisuccinate;
DPPG-Na: 1,2-Dipalmitoyl-sn-glycero-3-phosphoglycerol, sodium salt;
POPC: 1-palmitoyl-2-oleoyl-sn-glycero-3-phosphocholine;
DPPC: 1,2-dipalmitoyl-sn-glycero-3-phosphocholine;
DlinPC: 1,2-dilinoleoyl-sn-glycero-3-phosphocholine;
DOPE: 1,2-dioleoyl-sn-glycero-3-phosphoethanolamine;
NAPE: N-acetylphosphatidylethanolamine;
DODAP: 1,2-dioleoyl-3-dimethylammonium-propane

Uptake in Healthy Cells

To compare cellular uptake of liposomal formulations by phagocytic cells found in lungs, in vitro uptake of particles by macrophages was measured. Prior to uptake assays, THP-1 monocytes were differentiated into macrophages by 24-hour incubation with 50 ng/mL phorbol myristate acetate (PMA), followed by 24-hour incubation in fresh RPMI media. For uptake assays, differentiated macrophages cultured in Opti-MEM media containing 5% fetal bovine serum (FBS) were incubated with AF647-labeled particles (final lipid concentration of 140 μg/mL), gently harvested, and washed with phosphate-buffered saline (PBS). Particle uptake into individual cells was quantified by fluorescence-activated cell sorting (FACS) and normalized to the total amount of fluorescent label added per mL of media to calculate the normalized mean fluorescence intensity (MFI).

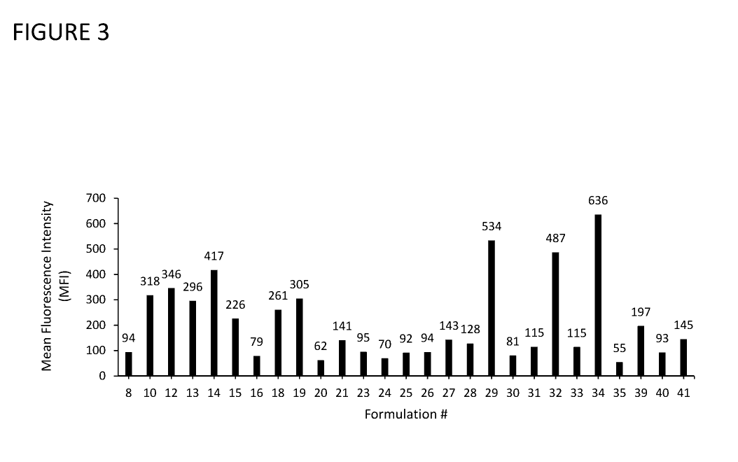

High uptake into macrophages with MFI values above 400 occurred with formulations 14, 29, 32, and 34 (see Table 5 for lipid components), composed of high concentrations of either CHEMS or THS (Table 6, FIG. 3). Other formulations containing various molar ratios of DOPC, CHEMS, THS, and DPPG-Na (formulations 10, 12, 13, 19, 21, 27, 28, 31, and 41 of Table 5) or POPE, Chol, THS, and CHEMS (formulations 15, 33, and 39 of Table 5) also exhibited good uptake (above MFI 100) into both macrophages and fibroblasts (Table 6, FIG. 3).

TABLE 6
Summary of uptake of fluorescently labeled liposome
formulations after 4 hour incubation with THP-1 cells

| Formulation # | Uptake levels (MFI) |
|---|---|
| 8 | 94 |
| 10 | 318 |
| 12 | 346 |
| 13 | 296 |
| 14 | 417 |
| 15 | 226 |
| 16 | 79 |
| 18 | 261 |
| 19 | 305 |
| 20 | 62 |
| 21 | 141 |
| 23 | 95 |
| 24 | 70 |
| 25 | 92 |
| 26 | 94 |
| 27 | 143 |
| 28 | 128 |
| 29 | 534 |
| 30 | 81 |
| 31 | 115 |
| 32 | 487 |
| 33 | 115 |
| 34 | 636 |
| 35 | 55 |
| 39 | 197 |
| 40 | 93 |
| 41 | 145 |

In Vitro Efficacy

Several liposomal amikacin formulations were loaded with antibiotic and evaluated for ability to reduce intracellular CFU of NTM abscessus-infected THP-1 cells.

THP-1 cells were seeded in PMA and cultured for 48 hours, exposed to M. abscessus (type strain 19977 or clinical isolate Stanford A or NIH26) at multiplicity of infection (MOI) of approx. 2 for 1 hour followed by incubation with 50 mg/mL amikacin sulfate for 23 hours to reduce the extracellular NTM population. Liposomal amikacin formulations were added at 16, 32, 64, and 128 μg/mL of active concentration and incubated for 24 hours at 37° C. At the end of four 24 hour treatment cycles, cells were washed, lysed, serially diluted, plated, and incubated at 37° C. for 4 days to determine final intracellular CFU counts. CFU numbers are shown in Table 7.

TABLE 7
Summary of remaining intracellular CFUs after 4 day treatment with amikacin-loaded liposomes

| | | | | Pretreatment + liposomal | | | | | |
|---|---|---|---|---|---|---|---|---|---|
| | | | | treatment | | | In vitro killing (additional | | |
| | CFU | | | In vitro killing (additional % | | | % CFU killed compared to | | |
| | In vitro killing (CFU remaining | | | CFU killed compared to | | | free ami treatment after | | |
| | after 96 h treatment with 128 | | | pretreatment after 96 h | | | 96 h treatment with 128 μg/ | | |
| Formulation # | μg/mL ami-in THP-1 NTM | | | treatment with 128 μg/mL | | | mL ami-in THP-1 NTM | | |
| (see Table 5 | model) | | | ami in THP-1 NTM model) | | | model) | | |
| for lipid | | Stanford | | | Stanford | | | Stanford | |
| components) | 19977 | A | NIH 26 | 19977 | A | NIH 26 | 19977 | A | NIH 26 |
| 8 | 2710 | 2000 | 4917 | 99 | 99.6 | 95.5 | 98.0 | 98.4 | 93.0 |
| 14 | 2513 | — | 4249 | 99.3 | — | 94.4 | 98.5 | — | 90.3 |
| 15 | 1660 | 200 | 3567 | 99.6 | 99.97 | 95.7 | 99.4 | 99.9 | 93.8 |
| 24 | 5600 | 8200 | — | 98.9 | 98.3 | — | 95.7 | 93.7 | — |
| 25 | 7200 | 9000 | — | 98.5 | 98.1 | — | 94.5 | 93.1 | — |
| 26 | 4000 | 3800 | — | 99.2 | 99.2 | — | 96.9 | 97.1 | — |
| 30 | 20000 | 8000 | — | 97.9 | 98.5 | — | 96.0 | 93.5 | — |
| 31 | 10557 | 6000 | 8937 | 97.7 | 98.8 | 91.2 | 96.3 | 95.2 | 86.3 |
| 33 | 2667 | 1400 | 4303 | 99.3 | 99.8 | 95.6 | 98.8 | 99.1 | 93.4 |
| 35 | 14437 | 4200 | 13050 | 96.7 | 99.1 | 85.5 | 94.8 | 96.8 | 76.8 |
| 39 | 2243 | 1200 | 3717 | 99.5 | 99.8 | 96.5 | 99.3 | 99.1 | 94.4 |
| free ami | 135000 | 135000 | 214667 | 50.1 | 75 | 37.4 | N/A | N/A | N/A |

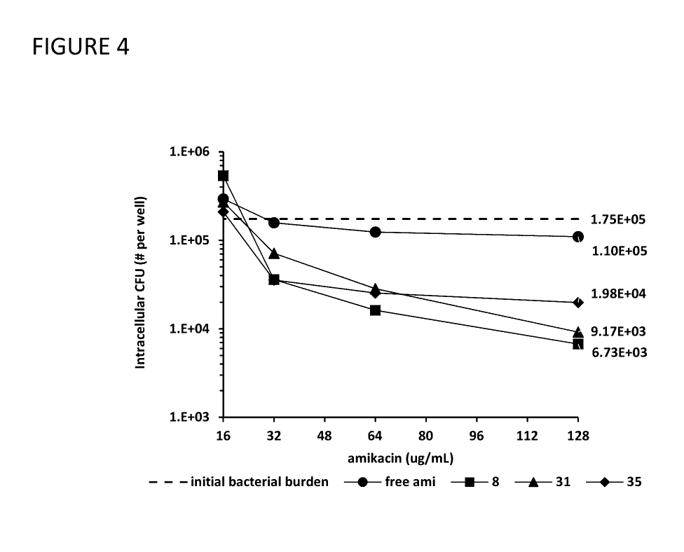

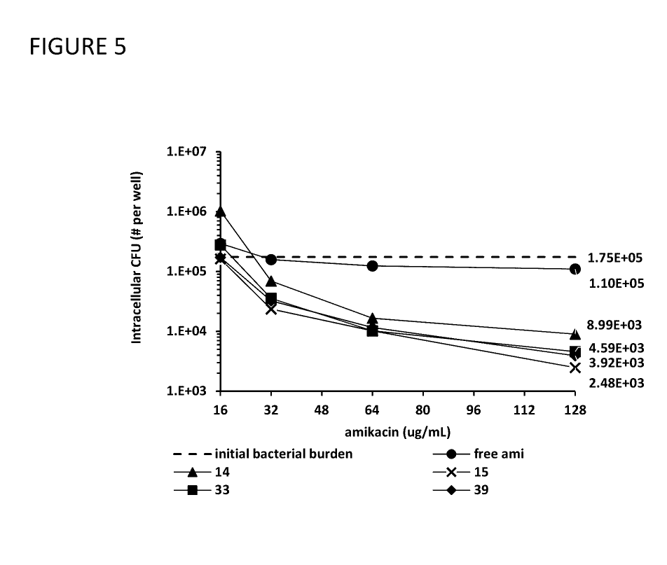

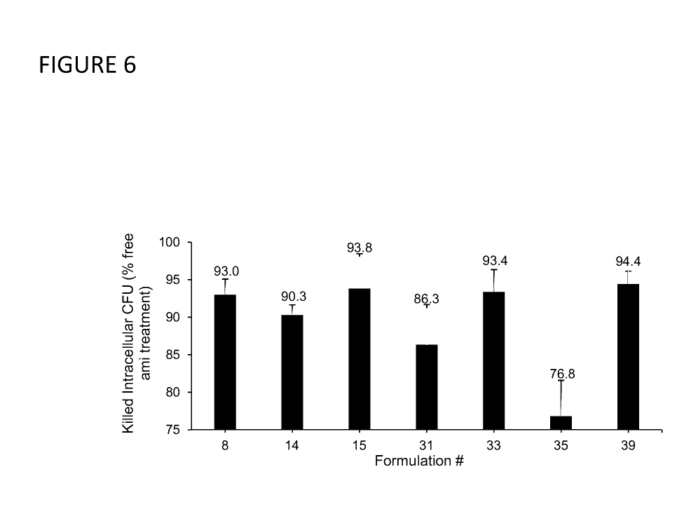

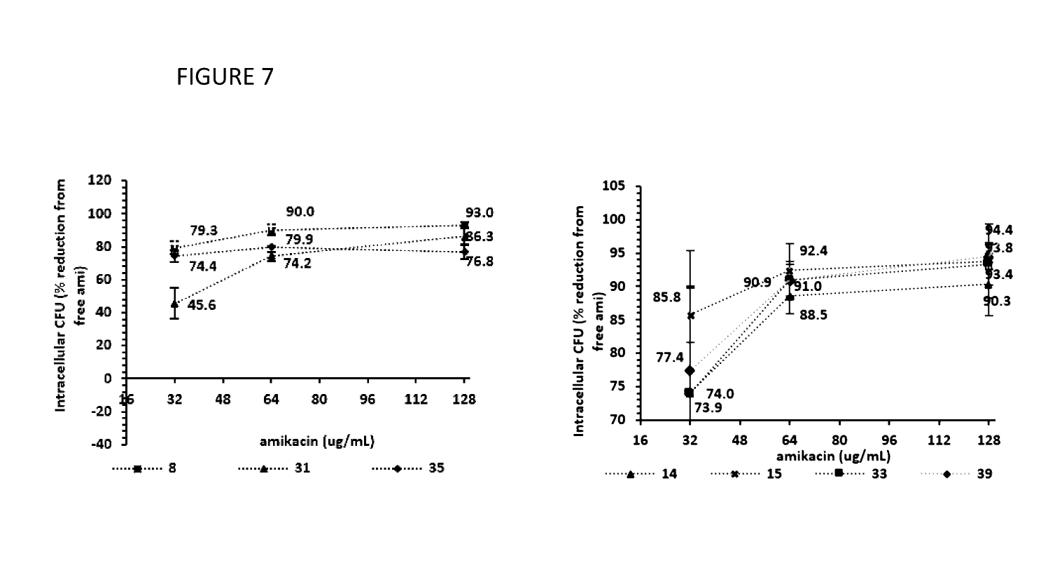

All formulations tested reduced CFUs by 96% or more compared to pretreatment levels in the M. ab. 19977 infected THP-1 cells, by 98.5% or more in the M. ab. Stanford A infected THP-1 cells, and by 85% or more in the M. ab. NIH26 infected THP-1 cells. The highest killing efficiency was recorded for formulation #s 8, 15, 33, and 39 (see Table 5 for lipid components). The NIH26 infection model results are plotted in FIGS. 4-7 (see Table 5 for lipid components of the formulations).

### Example 3—In Vitro Toxicity of Liposomal Amikacin Formulations

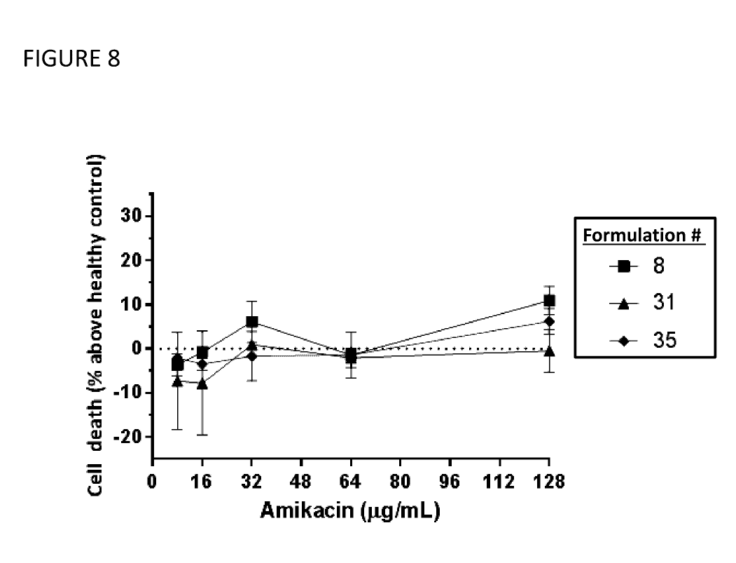

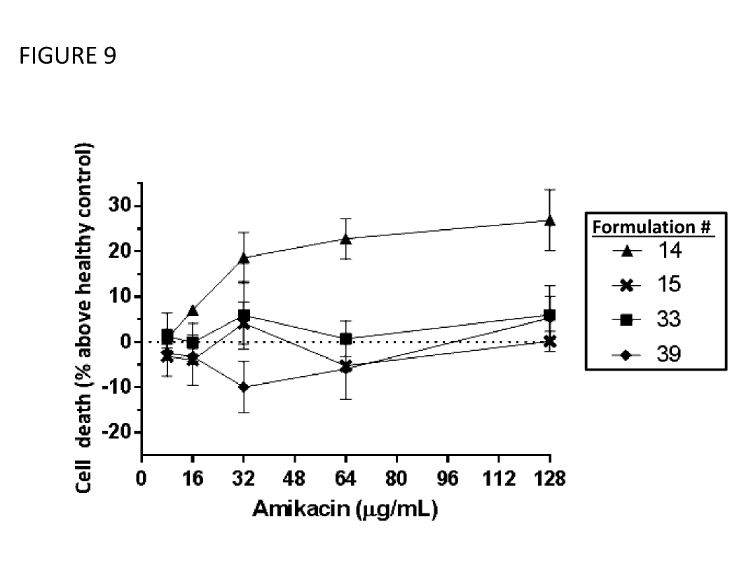

To evaluate toxicity of liposomal amikacin formulations, differentiated healthy THP-1 cells were treated 4× (1× every 24 hours) with 16, 32, 64, or 128 μg/mL amikacin concentrations for the seven liposomal amikacin formulations 8, 14, 15, 31, 33, 35, 39 (Table 5 for lipid components). Formulation 14 increases cell death >25% at 128 μg/mL compared to no treatment control, formulation 8 increases cell death 11% at 128 μg/mL, formulation 15 and formulation 31 show no difference in cell death compared to no treatment control, and formulations 33 and 39 fall in between formulation 8 and formulation 15 cell death levels (FIGS. 8 and 9).

### Example 4—In Vitro Characterization of Liposomal Streptomycin Formulations

Streptomycin sulfate liposomal formulations were manufactured via an in-line infusion process. The lipid stream was infused at a lipid concentration of 20 mg/mL to 80 mg/mL at a flow rate of 10 mL/min to about 25 mL/min, and the aqueous amikacin sulfate stream was infused at an streptomycin sulfate concentration of 80 mg/mL to about 200 mg/mL at a flow rate of 15 mL/min to 40 mL/min. After infusion, the products were washed to remove streptomycin amikacin sulfate using tangential flow filtration (TFF).

Formulations were loaded with streptomycin and tested against the M. abscessus strains 19977 and Stanford A. Results are shown in Table 8.

TABLE 8

| | | | In vitro killing (addtl. % | | In vitro killing (addtl. | |
|---|---|---|---|---|---|---|
| | In vitro killing (CFU | | CFU killed compared to | | % CFU killed compared | |
| | remaining after 96 h | | pretreatment after 96 h | | to free ami treatment after | |
| | treatment with 128 μg/ | | treatment with 128 μg/mL | | 96 h treatment with 128 | |
| | mL streptomycin sulfate | | streptomycin sulfate in | | μg/mL streptomycin sulfate | |
| Formulation | in THP-1 NTM model) | | THP-1 NTM model) | | in THP-1 NTM model) | |
| # | 19977 | Stanford A | 19977 | Stanford A | 19977 | Stanford A |
| 8 | 316000 | 160000 | 50.3 | 72.6 | 22.5 | 62.3 |
| 14 | 104000 | 64000 | 83.6 | 89.0 | 74.5 | 84.9 |
| 15 | 172000 | 116000 | 73.0 | 80.1 | 57.8 | 72.6 |
| free | 408000 | 424000 | 35.8 | 27.4 | N/A | N/A |
| streptomycin | | | | | | |
| sulfate | | | | | | |

All, documents, patents, patent applications, publications, product descriptions, and protocols which are cited throughout this application are incorporated herein by reference in their entireties for all purposes.

The embodiments illustrated and discussed in this specification are intended only to teach those skilled in the art the best way known to the inventors to make and use the invention. Modifications and variation of the above-described embodiments of the invention are possible without departing from the invention, as appreciated by those skilled in the art in light of the above teachings. It is therefore understood that, within the scope of the claims and their equivalents, the invention may be practiced otherwise than as specifically described. Accordingly, the foregoing descriptions and drawings are by way of example only and the disclosure is described in detail by the claims that follow.

1. A pharmaceutical composition comprising an antibiotic encapsulated in liposomes, wherein the lipid component of the liposomes comprises an unsaturated phospholipid, and the antibiotic-to-lipid component weight ratio is 0.5 (antibiotic)-to-1 (lipid component) or greater.

2. The pharmaceutical composition of claim 1, wherein the unsaturated phospholipid is an unsaturated phosphatidylethanolamine (PE).

3. The pharmaceutical composition of claim 1, wherein the unsaturated phospholipid is 1,2-dioleoyl-sn-glycero-3-phosphocholine (DOPC).

4. The pharmaceutical composition of claim 1, wherein the unsaturated phospholipid is 1-palmitoyl-2-oleoyl-sn-glycero-3-phosphocholine (POPC).

5. The pharmaceutical composition of claim 2, wherein the unsaturated PE is dioleoylphosphatidylethanolamine (DOPE), N-acyl phosphatidylethanolamine (NAPE) or 1-palmitoyl-2-oleoyl-sn-glycero-3-phosphoethanolamine (POPE).

6. The pharmaceutical composition of claim 5, wherein the unsaturated PE is dioleoylphosphatidylethanolamine (DOPE).

7. The pharmaceutical composition of claim 5, wherein the unsaturated PE is N-acyl phosphatidylethanolamine (NAPE)

8. The pharmaceutical composition of claim 5, wherein the unsaturated PE is 1-palmitoyl-2-oleoyl-sn-glycero-3-phosphoethanolamine (POPE).

9. The pharmaceutical composition of any one of claims 1-8, wherein the lipid component of the liposomes further comprises cholesterol.

10. The pharmaceutical composition of any one of claims 1-9, wherein the lipid component of the liposomes further comprises D-α-tocopherol-hemisuccinate (THS).

11. The pharmaceutical composition of any one of claims 1-10, wherein the lipid component of the liposomes further comprises cholesteryl hemi succinate (CHEMS).

12. The pharmaceutical composition of any one of claims 1-10, wherein the lipid component of the liposomes further comprises 1,2-Dipalmitoyl-sn-glycero-3-phosphoglycerol sodium (DPPG-Na).

13. The pharmaceutical composition of any one of claims 4-12, wherein the lipid component of the liposomes further comprises 1,2-dioleoyl-sn-glycero-3-phosphocholine (DOPC).

14. The pharmaceutical composition of claim 1, wherein the lipid component of the liposomes consists of 1,2-dioleoyl-sn-glycero-3-phosphocholine (DOPC).

15. The pharmaceutical composition of claim 1, wherein the lipid component of the liposomes consists of 1-palmitoyl-2-oleoyl-sn-glycero-3-phosphocholine (POPC).

16. The pharmaceutical composition of claim 1, wherein the lipid component of the liposomes consists of 1,2-dioleoyl-sn-glycero-3-phosphocholine (DOPC) and 1,2-Dipalmitoyl-sn-glycero-3-phosphoglycerol sodium (DPPG-Na).

17. The pharmaceutical composition of claim 1, wherein the lipid component of the liposomes consists 1-palmitoyl-2-oleoyl-sn-glycero-3-phosphoethanolamine (POPE), cholesterol and 1,2-dioleoyl-sn-glycero-3-phosphocholine (DOPC).

18. The pharmaceutical composition of claim 1, wherein the lipid component of the liposomes consists of 1-palmitoyl-2-oleoyl-sn-glycero-3-phosphoethanolamine (POPE), cholesterol, D-α-tocopherol-hemisuccinate (THS) and 1,2-dioleoyl-sn-glycero-3-phosphocholine (DOPC).

19. The pharmaceutical composition of claim 1, wherein the lipid component of the liposomes consists of 1-palmitoyl-2-oleoyl-sn-glycero-3-phosphoethanolamine (POPE), cholesteryl hemi succinate (CHEMS) and 1,2-dioleoyl-sn-glycero-3-phosphocholine (DOPC).

20. The pharmaceutical composition of claim 16, wherein the molar ratio of 1,2-dioleoyl-sn-glycero-3-phosphocholine (DOPC) to 1,2-Dipalmitoyl-sn-glycero-3-phosphoglycerol sodium (DPPG-Na) is about 5 (DOPC):about 1 (DPPG-Na) to about 12 (DOPC):about 1 (DPPG-Na).

21. The pharmaceutical composition of claim 16, wherein the molar ratio of 1,2-dioleoyl-sn-glycero-3-phosphocholine (DOPC) to 1,2-Dipalmitoyl-sn-glycero-3-phosphoglycerol sodium (DPPG-Na) is about 9 (DOPC):about 1 (DPPG-Na).

22. The pharmaceutical composition of claim 17, wherein the molar ratio of 1-palmitoyl-2-oleoyl-sn-glycero-3-phosphoethanolamine (POPE) to cholesterol to 1,2-dioleoyl-sn-glycero-3-phosphocholine (DOPC) is from about 1 (POPE):about 4 (cholesterol):5 (DOPC) to about 5 (POPE):about 2 (cholesterol):about 3 (DOPC).

23. The pharmaceutical composition of claim 17, wherein the molar ratio of 1-palmitoyl-2-oleoyl-sn-glycero-3-phosphoethanolamine (POPE) to cholesterol to 1,2-dioleoyl-sn-glycero-3-phosphocholine (DOPC) is about 6 (POPE):about 7.5 (cholesterol):about 10 (DOPC).

24. The pharmaceutical composition of claim 18, wherein the molar ratio of 1-palmitoyl-2-oleoyl-sn-glycero-3-phosphoethanolamine (POPE), to cholesterol to D-α-tocopherol-hemisuccinate (THS) to 1,2-dioleoyl-sn-glycero-3-phosphocholine (DOPC) is from about 1 (POPE):about 1 (Chol):about 0.5 (THS):about 1 (DOPC) to about 9 (POPE):about 9 (Chol):5 (THS):about 9 (DOPC).

25. The pharmaceutical composition of claim 18, wherein the molar ratio of 1-palmitoyl-2-oleoyl-sn-glycero-3-phosphoethanolamine (POPE), to cholesterol to D-α-tocopherol-hemisuccinate (THS) to 1,2-dioleoyl-sn-glycero-3-phosphocholine (DOPC) is about 4 (POPE):about 4 (Chol):about 1 (THS):about 2.5 (DOPC).

26. The pharmaceutical composition of claim 18, wherein the molar ratio of 1-palmitoyl-2-oleoyl-sn-glycero-3-phosphoethanolamine (POPE), to cholesterol to D-α-tocopherol-hemisuccinate (THS) to 1,2-dioleoyl-sn-glycero-3-phosphocholine (DOPC) is about 6 (POPE):about 6 (Chol):about 1.5 (THS):about 10 (DOPC).

27. The pharmaceutical composition of claim 19, wherein the molar ratio of 1-palmitoyl-2-oleoyl-sn-glycero-3-phosphoethanolamine (POPE) to cholesteryl hemisuccinate (CHEMS) to 1,2-dioleoyl-sn-glycero-3-phosphocholine (DOPC) is from about 1 (POPE):about 1 (CHEMS):about 1 (DOPC) to about 5 (POPE):about 1 (CHEMS):about 5 (DOPC).

28. The pharmaceutical composition of claim 19, wherein the molar ratio of 1-palmitoyl-2-oleoyl-sn-glycero-3-phosphoethanolamine (POPE) to cholesteryl hemisuccinate (CHEMS) to 1,2-dioleoyl-sn-glycero-3-phosphocholine (DOPC) is about 4 (POPE):1 (CHEMS):4 (DOPC).

29. The pharmaceutical composition of claim 1, wherein the lipid component of the liposomes is selected from one of the following:

| | | |
|---|---|---|
| PE/CHEMS; PE/PC/CHEMS | DOPE/CHEMS/PE-PEG | DOPE/Chol |
| PE/Chol | DOPE/CHEMS/Chol | POPE/Chol |
| PE/THS | DOPE/DODAP/DOPC | NAPE/Chol |
| PE/CHEMS/Chol | NAPE/DODAP/DOPC | DOPE/Chol/THS |
| DOPE/OA/Chol | POPE/DODAP/DOPC | POPE/Chol/THS |
| DOPE/CHEMS | DOPE/DOPS/PEG- | NAPE/Chol/THS |
| | ceramide; | |
| | NAPE/DOPS/PEG- | |
| | ceramide | |
| NAPE/CHEMS | POPE/DOPS/PEG- | POPE/CHEMS |
| | ceramide | |
| DOPE/N-succinyl-DOPE | DOPE/N-glutaryl- | DOPE/N-glutaryl- |
| | DOPE | DOPE/PEG-ceramide |
| DOPE/N-succinyl-DOPE/ | NAPE/N-glutaryl- | DOPE/N-glutaryl- |
| PEG-ceramide | DOPE | DOPE/Chol/PEG-ceramide |
| DOPE/N-succinyl-DOPE/ | POPE/N-glutaryl-DOPE | PC/CHEMS/Tween-80/ |
| Cholesterol/PEG-ceramide | | OAlc |
| DOPE/DSPG | POPE/DOSG | EPC/DDAB/CHEMS/Tween-80 |
| DOPE/DOSG | DOPE/HSPC/CHEMS/Chol | PC/DDAB/CHEMS/Tween-80 |
| NAPE/DOSG | DOPE/HSPC/CHEMS/Chol | PC/CHEMS/Tween-80/ |
| | | OAlc |
| DOPE/N-citraconyl-DOPE/Chol | POPE/N-citraconyl- | POPE/Chol/MPL |
| | DOPE/Chol | |
| NAPE/N-citraconyl-DOPE/Chol | DDAB/CHEMS | YSK05/POPE/Cholesterol/ |
| | | DMG-PEG |
| Diolein/CHEMS | EPC/CHEMS/T-80/OAlc | EPC/CHEMS/DDAB/T-80 |
| PE/PC/CHEMS | | |

30. The pharmaceutical composition of any one of claims 1-29, wherein the antibiotic-to-lipid component weight ratio in the composition is about 0.5 (antibiotic):1 (lipid component) or greater, about 1 (antibiotic):1 (lipid component) or greater, about 1.5 (antibiotic):1 (lipid component) or greater, about 2 (antibiotic):1 (lipid component) or greater or about 2.5 (antibiotic):1 (lipid component) or greater.

31. The pharmaceutical composition of any one of claims 1-30, wherein the antibiotic-to-lipid component weight ratio in the composition is from about 0.5-to-1 (antibiotic-to-lipid component) to about 3-to-1 (antibiotic-to-lipid component).

32. The pharmaceutical composition of any one of claims 1-30, wherein the antibiotic-to-lipid component weight ratio in the composition is from about 1-to-1 (antibiotic-to-lipid component) to about 3-to-1 (antibiotic-to-lipid component).

33. The pharmaceutical composition of any one of claims 1-30, wherein the antibiotic-to-lipid component weight ratio in the composition is from about 1.5-to-1 (antibiotic-to-lipid component) to about 3-to-1 (antibiotic-to-lipid component).

34. The pharmaceutical composition of any one of claims 1-30, wherein the antibiotic-to-lipid component weight ratio in the composition is from about 2-to-1 (antibiotic-to-lipid component) to about 3-to-1 (antibiotic-to-lipid component).

35. The pharmaceutical composition of any one of claims 1-34, wherein the antibiotic is an aminoglycoside or a pharmaceutically acceptable salt thereof.

36. The pharmaceutical composition of claim 35, wherein the aminoglycoside is amikacin, apramycin, arbekacin, astromicin, capreomycin, dibekacin, framycetin, gentamicin, hygromycin B, isepamicin, kanamycin, neomycin, netilmicin, paromomycin, rhodestreptomycin, ribostamycin, sisomicin, spectinomycin, streptomycin, tobramycin, verdamicin, a pharmaceutically acceptable salt thereof, or a combination thereof.

37. The pharmaceutical composition of claim 35, wherein the aminoglycoside is AC4437, amikacin, apramycin, arbekacin, astromicin, bekanamycin, boholmycin, brulamycin, capreomycin, dibekacin, dactimicin, etimicin, framycetin, gentamicin, H107, hygromycin, hygromycin B, inosamycin, K-4619, isepamicin, KA-5685, kanamycin, neomycin, netilmicin, paromomycin, plazomicin, ribostamycin, sisomicin, rhodestreptomycin, sorbistin, spectinomycin, sporaricin, streptomycin, tobramycin, verdamicin, vertilmicin, or a pharmaceutically acceptable salt thereof.

38. The pharmaceutical composition of claim 35, wherein the aminoglycoside is amikacin, or a pharmaceutically acceptable salt thereof.

39. The pharmaceutical composition of claim 38, wherein the pharmaceutically acceptable salt of amikacin is amikacin sulfate.

40. The pharmaceutical composition of claim 35, wherein the aminoglycoside is streptomycin, or a pharmaceutically acceptable salt thereof.

41. The pharmaceutical composition of claim 40, wherein the pharmaceutically acceptable salt of streptomycin is streptomycin sulfate.

42. The pharmaceutical composition of any one of claims 1-41, further comprising a free antibiotic.

43. The pharmaceutical composition of claim 42, wherein the ratio by weight of free antibiotic to the antibiotic encapsulated in the liposomes is from about 1:100 to about 100:1.

44. The pharmaceutical composition of claim 42, wherein the ratio by weight of free antibiotic to the antibiotic encapsulated in the liposomes is from about 1:50 to about 50:1.

45. The pharmaceutical composition of claim 42, wherein the ratio by weight of free antibiotic to the antibiotic encapsulated in the liposomes is from about 1:10 to about 10:1.

46. The pharmaceutical composition of claim 42, wherein the ratio by weight of free antibiotic to the antibiotic encapsulated in the liposomes is from about 1:5 to about 5:1.

47. The pharmaceutical composition of claim 42, wherein the ratio by weight of free antibiotic to the antibiotic encapsulated in the liposomes is from about 1:4 to about 4:1.

48. The pharmaceutical composition of claim 42, wherein the ratio by weight of free antibiotic to the antibiotic encapsulated in the liposomes is from about 1:3 to about 3:1.

49. The pharmaceutical composition of claim 42, wherein the ratio by weight of free antibiotic to the antibiotic encapsulated in the liposomes is from about 1:2 to about 2:1.

50. The pharmaceutical composition of any one of claims 42-49, wherein the free antibiotic is a free aminoglycoside or a pharmaceutically acceptable salt thereof.

51. The pharmaceutical composition of claim 50, wherein the free aminoglycoside is amikacin, apramycin, arbekacin, astromicin, capreomycin, dibekacin, framycetin, gentamicin, hygromycin B, isepamicin, kanamycin, neomycin, netilmicin, paromomycin, rhodestreptomycin, ribostamycin, sisomicin, spectinomycin, streptomycin, tobramycin, verdamicin, a pharmaceutically acceptable salt thereof, or a combination thereof.

52. The pharmaceutical composition of claim 50, wherein the free aminoglycoside is AC4437, amikacin, apramycin, arbekacin, astromicin, bekanamycin, boholmycin, brulamycin, capreomycin, dibekacin, dactimicin, etimicin, framycetin, gentamicin, H107, hygromycin, hygromycin B, inosamycin, K-4619, isepamicin, KA-5685, kanamycin, neomycin, netilmicin, paromomycin, plazomicin, ribostamycin, sisomicin, rhodestreptomycin, sorbistin, spectinomycin, sporaricin, streptomycin, tobramycin, verdamicin, vertilmicin, or a pharmaceutically acceptable salt thereof.

53. The pharmaceutical composition of claim 50, wherein the free aminoglycoside is free amikacin, or a pharmaceutically acceptable salt thereof.

54. The pharmaceutical composition of claim 53, wherein the free pharmaceutically acceptable salt of amikacin is amikacin sulfate.

55. The pharmaceutical composition of claim 50, wherein the free aminoglycoside is free streptomycin, or a pharmaceutically acceptable salt thereof.

56. The pharmaceutical composition of claim 55, wherein the free pharmaceutically acceptable salt of streptomycin is streptomycin sulfate.

57. A system comprising the pharmaceutical composition of any one of claims 1-56 and a nebulizer.

58. The pharmaceutical composition of any one of claims 1-56, wherein the composition is an aerosol.

59. A method for treating a bacterial infection or a disease associated with an intracellular bacterial infection in a patient in need thereof, comprising administering to the patient, an effective amount of the pharmaceutical composition of any one of claims 1-56.

60. The method of claim 59, wherein the administering to the patient comprises parenteral administration.

61. The method of claim 60, wherein the parenteral administration comprises intravenous, intramuscular or subcutaneous administration.

62. The method of claim 58, wherein the administering to the patient comprises inhalation administration.

63. The method of claim 62, wherein inhalation administration is conducted via a nebulizer.

64. The method of claim 62, wherein inhalation administration is conducted via a dry powder inhaler (DPI).

65. The method of any one of claims 59-64, wherein the bacterial infection is a Rhodococcus infection.

66. The method of claim 65, wherein the Rhodococcus infection is a R. equi infection.

67. The method of claim 65, wherein the Rhodococcus infection is a R. fascians infection.

68. The method of any one of claims 59-64, wherein the bacterial infection is a Salmonella Listeria, Francisella, Streptobacillus, Trypanosoma, Entamoeba, Cryptosporidium, Coxiella burnetii, Streptococcus, Porphyromonas, Eikenella corrodens, Prevotella, Chlamydia, Tannerella forsythia, Treponema, Mycoplasma, Yersina, Corynebacterium or a Borrelia infection.

69. The method of claim 68, wherein the bacterial infection is a Salmonella infection.

70. The method of claim 69, wherein the Salmonella infection is a Salmonella typhimurium or a Salmonella typhi infection.

71. The method of claim 68, wherein the bacterial infection is a Listeria infection.

72. The method of claim 71, wherein the Listeria infection is a Listeria monocytogens infection.

73. The method of claim 68, wherein the bacterial infection is a Yersina infection.

74. The method of claim 73, wherein the Yersina infection is a Y. pestis, Y. aldovae, Y. aleksiciae, Y. bercovien, Y. enterocolitica, Y. entomophaga, Y. frederiksenii, Y. intermdia, Y. kristensenii, Y. massiliensis, Y. mollaretii, Y. nurmii, Y. pekkanenii, Y. philomiragia, Y. pseudotuberculosis, Y. rohdei, Y. ruckeri or a Y. similis infection.

75. The method of claim 68, wherein the bacterial infection is a Streptobacillus infection.

76. The method of claim 75, wherein the Streptobacillus infection is a Streptobacillus moniliformis infection.

77. The method of claim 68, wherein the bacterial infection is an Entamoeba infection.

78. The method of claim 77, wherein the Entamoeba infection is an Entamoeba histolytica or an Entamoeba dispar infection.

79. The method of claim 68, wherein the bacterial infection is a Mycoplasma infection.

80. The method of claim 79, wherein the Mycoplasma infection is a M. genitalium or a M. pneumoniae infection.

81. The method of claim 68, wherein the bacterial infection is a Prevotella infection.

82. The method of claim 81, wherein the Prevotella infection is a Prevotella melaninogenica or a Prevotella intermedia infection.

83. The method of claim 68, wherein the bacterial infection is a Chlamydia infection.

84. The method of claim 83, wherein the Chlamydia infection is a Chlamydia trachomatis infection.

85. The method of claim 68, wherein the bacterial infection is a Treponema infection.

86. The method of claim 85, wherein the Treponema infection is a Treponema denticola, Treponema palladium or a Treponema carateum infection.

87. The method of claim 68, wherein the bacterial infection is a Streptococcus infection.

88. The method of claim 87, wherein the Streptococcus infection is a Streptococcal L-form, S. mutans, S. pyogenes or a S. agalactiae infection.

89. The method of claim 68, wherein the bacterial infection is a Porphyromonas infection.

90. The method of claim 89, wherein the Porphyromonas infection is a P. gingivalis infection.

91. The method of claim 68, wherein the bacterial infection is a Cryptosporidium infection.

92. The method of claim 91, wherein the Cryptosporidium infection is a Cryptosporidium parvum infection.

93. The method of any one of claims 59-64, wherein the bacterial infection is Shigellae infection.

94. The method of claim 93, wherein the Shigellae infection is a S. boydii, S. dysenteriae, S. flexneri or a S. sonnei infection.

95. The method of any one of claims 59-64, wherein the bacterial infection is a L. pneumophila infection.

96. The method of any one of claims 59-64, wherein the bacterial infection is a Rickettsia infection.

97. The method of any one of claims 59-64, wherein the bacterial infection is a Legionella infection.

98. The method of claim 97, wherein the Legionella infection is a L. pneumophila, L. longbeachae, L. feeleii, L. micdadei or a L. anisa infection.

99. The method of any one of claims 59-64, wherein the bacterial infection is a mycobacterial infection.

100. The method of claim 99, wherein the mycobacterial infection is a M. tuberculosis infection.

101. The method of claim 100, wherein the M. tuberculosis is multi-drug resistant.

102. The method of claim 100, wherein the patient in need of treatment has Vank's disease.

103. The method of claim 99, wherein the mycobacterial infection is a M. leprae infection.

104. The method of claim 99, wherein the mycobacterial infection is a nontuberculous mycobacterial (NTM) infection.

105. The method of claim 104, wherein the NTM infection is an NTM lung infection.

106. The method of claim 105, wherein the NTM lung infection is a M. avium, M. avium subsp. hominissuis (MAH), M. abscessus, M. chelonae, M. bolletii, M. xenopi, M. massiliense, M. kansasii, M. ulcerans, M. avium, M. avium complex (MAC) (M. avium and M. intracellulare), M. conspicuum, M. peregrinum, M. immunogenum, M. marinum, M. malmoense, M. mucogenicum, M. nonchromogenicum, M. scrofulaceum, M. simiae, M. smegmatis, M. szulgai, M. terrae, M. terrae complex, M. haemophilum, M. genavense, M. asiaticum, M. shimoidei, M. gordonae, M. nonchromogenicum, M. triplex, M. lentiflavum, M. celatum, M. fortuitum, M. fortuitum complex (M. fortuitum and M. chelonae) lung infection, or a combination thereof.

107. The method of claim 105, wherein the NTM lung infection is a Mycobacterium abscessus lung infection.

108. The method of claim 105, wherein the NTM lung infection is Mycobacterium avium complex (M. avium and M. intracellulare) lung infection.

109. The method of claim 105, wherein the NTM lung infection is a M. kansasii lung infection.

110. The method of claim 105, wherein the NTM lung infection is a M. fortuitum lung infection.

111. The method of claim 105, wherein the NTM lung infection is a M. chelonae lung infection.

112. The method of claim 105, wherein the NTM lung infection is a M. xenopi lung infection.

113. The method of claim 105, wherein the NTM lung infection is a M. simiae lung infection.

114. The method of claim 105, wherein the NTM lung infection is a M. massiliense lung infection.

115. The method of any one of claims 105-114, wherein the NTM lung infection is an NTM lung infection with a presentation similar to hypersensitivity lung disease.

116. The method of any one of claims 105-115, wherein the NTM lung infection is a macrolide resistant NTM lung infection.

117. The method of any one of claims 59-116, wherein the encapsulated antibiotic is an aminoglycoside or a pharmaceutically acceptable salt thereof, and the administering comprises inhalation administration.

118. The method of claim 117, wherein the encapsulated aminoglycoside or pharmaceutically acceptable salt thereof is amikacin sulfate.

119. The method of any one of claims 59-118, wherein the effective amount of the pharmaceutical composition is administered once daily or every other day during an administration period.

120. The method of claim 119, wherein during the administration period or subsequent to the administration period, the patient experiences a change from baseline on the full semi quantitative scale for mycobacterial culture and/or NTM culture conversion to negative.

121. The method of claim 119 or 120, wherein during the administration period or subsequent to the administration period, the patient exhibits an increased number of meters walked in the 6 minute walk test (6MWT), as compared to the number of meters walked by the patient prior to the administration period, or a greater number of meters walked in the 6MWT, as compared to a patient subjected to a non-liposomal aminoglycoside treatment for the NTM lung infection.

122. The method of any one of claims 118-121, wherein the patient experiences an improvement in FEV₁ for at least 15 days after the administration period ends, as compared to the FEV₁ of the patient prior to treatment.

123. The method of any one of claims 59-122, wherein the effective amount of the composition comprises from about 100 mg to about 1000 mg aminoglycoside, or pharmaceutically acceptable salt thereof, or from about 200 mg to about 900 mg aminoglycoside, or pharmaceutically acceptable salt thereof, or from about 300 mg to about 800 mg aminoglycoside, or pharmaceutically acceptable salt thereof.

124. The method of any one of claims 59-123, wherein the effective amount of the composition is administered once per day in a single dosing session during an administration period.

125. The method of any one of claims 59-124, wherein the patient in need of treatment is a cystic fibrosis patient.

126. The method of any one of claims 59-125, wherein the patient in need of treatment is a bronchiectasis patient.

127. The method of any one of claims 59-126, wherein the patient in need of treatment is a smoker or has a previous history of smoking.

128. The method of any one of claims 59-127, wherein the patient in need of treatment has chronic obstructive pulmonary disorder (COPD).

129. The method of any one of claims 59-128, wherein the patient in need of treatment has asthma.

130. The method of any one of claims 104-129, wherein the patient in need of treatment was previously unresponsive to NTM therapy.

131. The method of any one of claims 59-130, wherein the patient in need of treatment or prophylaxis is a ciliary dyskinesia patient.

132. The method of any one of claims 59-131, wherein the patient in need of treatment has a co-morbid condition selected from diabetes, mitral valve disorder, acute bronchitis, pulmonary hypertension, pneumonia, asthma, trachea cancer, bronchus cancer, lung cancer, cystic fibrosis, pulmonary fibrosis, a larynx anomaly, a trachea anomaly, a bronchus anomaly, aspergillosis, HIV or bronchiectasis, in addition to the pulmonary NTM infection.

133. The method of claim 132, wherein the mitral valve disorder is mitral valve prolapse.

134. The method of any one of claims 59-133, further comprising administering to the patient in need of treatment, one or more additional therapeutic agents.

135. The method of any one of claims 59-134, wherein the patient's FEV₁ is increased at least 5% over the FEV₁ of the patient prior to the administration period.

136. The method of claim 135, wherein the patient's FEV₁ is increased at least 10% over the FEV₁ of the patient prior to the administration period.

137. The method of claim 135, wherein the patient's FEV₁ is increased at least 15% over the FEV₁ of the patient prior to the administration period.

138. The method of claim 135, wherein the patient's FEV₁ is increased by 5% to 50% over the FEV₁ prior to the administration period.

139. The method of any one of claims 104-138, wherein the patient exhibits an increased number of meters walked in the 6 minute walk test (6MWT), as compared to the number of meters walked by the patient prior to undergoing the treatment method.

140. The method of claim 139, wherein the increased number of meters walked in the 6MWT, in one embodiment, is at least about 5 meters.

141. The method of claim 139, wherein the increased number of meters walked in the 6MWT, in one embodiment, is at least about 10 meters.

142. The method of claim 139, wherein the increased number of meters walked in the 6MWT, in one embodiment, is at least about 20 meters.

143. The method of claim 139, wherein the increased number of meters walked in the 6MWT, in one embodiment, is at least about 30 meters.

144. The method of claim 139, wherein the increased number of meters walked in the 6MWT, in one embodiment, is at least about 40 meters.

145. The method of claim 139, wherein the increased number of meters walked in the 6MWT, in one embodiment, is at least about 50 meters.

146. The method of claim 139, wherein the increased number of meters walked in the 6MWT, in one embodiment, is from about 5 meters to about 50 meters.

147. The method of claim 139, wherein the increased number of meters walked in the 6MWT, in one embodiment, is from about 15 meters to about 50 meters.
